(19) United States
(12) Patent Application Publication
(10) Pub. No.: US 2010/0280118 A1
(43) Pub. Date: Nov. 4, 2010

(54) Methods for treating substance dependence

(75) Inventors: Barbara J. Mason, Rancho Santa Fe, CA (US); George F. Koob, Rancho Santa Fe, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 12/799,376

(22) Filed: Apr. 23, 2010

Related U.S. Application Data
(63) Continuation-in-part of application No. PCT/US08/12159, filed on Oct. 27, 2008.
(60) Provisional application No. 61/000,525, filed on Oct. 26, 2007.

(51) Int. Cl. A61K 31/197; A61K 31/195; A61P 25/30; A61P 25/32

(52) U.S. Cl. 514/561

(57) ABSTRACT

The present invention provides methods for treating alcohol dependence and preventing relapse in subjects in protracted alcohol abstinence. Also provided are methods for treating subjects suffering from cannabis dependence. Typically, these methods entail administering to the subjects in need of treatment a therapeutically effective amount of gabapentin, an analog of gabapentin, or pharmaceutically acceptable salt thereof.

### CROSS-REFERENCE TO RELATED APPLICATIONS

The subject patent application is a continuation-in-part application under 35 U.S.C. 111(a) of international application PCT/US2008/012159, filed Oct. 27, 2008, which in turn claims the benefit of priority to U.S. Provisional Patent Application No. 61/000,525 (filed Oct. 26, 2007). The full disclosures of the priority applications are incorporated herein by reference in their entirety and for all purposes.

### STATEMENT OF GOVERNMENT SUPPORT

This invention was made in part by government support by the National Institutes of Health Grant Nos. AA014028, DA020766 and AA012602. The U.S. Government therefore has certain rights in the invention.

### BACKGROUND OF THE INVENTION

Gabapentin (brand name Neurontin®) is a medication originally developed for the treatment of epilepsy. It is also widely used to relieve pain, especially neuropathic pain. Gabapentin is well tolerated in most patients, has a relatively mild side-effect profile, and passes through the body unmetabolized. Gabapentin was initially synthesized to mimic the chemical structure of the neurotransmitter gamma-aminobutyric acid (GABA), but is not believed to act on the same brain receptors. Its exact mechanism of action is unknown, but its therapeutic action on neuropathic pain is thought to involve voltage-gated N-type calcium ion channels. It is thought to bind to the α2δ subunit of the voltage-dependent calcium channel in the central nervous system.

Relapse prevention following detoxification has been identified as a leading concern of alcoholism research, with 90% of alcoholics estimated to relapse one or more times in the 4-year period following treatment. The first months following cessation of drinking represent the highest risk for relapse and offer the greatest opportunity for pharmacological intervention in outpatients with alcohol dependence. Historically, pharmacotherapy for alcohol-related problems has focused on treating acute withdrawal symptoms in the days immediately following the last drink, and a wide range of safe and effective medications are currently available for this indication. Fewer medications are available, however, to support abstinence post withdrawal. Recent advances in the neurobiology of alcohol dependence and factors implicated in relapse have helped identify medications potentially useful in post withdrawal treatment of alcoholism. These newer relapse-prevention medications, e.g., naltrexone, nalmefene, odansetron, topiramate, or acamprosate, have been found to be more effective than placebo for preventing drinking relapse in double-blind clinical trials. However, the effect size of all relapse prevention medications studied to date is modest.

Cannabis (i.e., marijuana and cannabinoids) is the most commonly used illicit drug in developed countries, and the lifetime prevalence of marijuana dependence is the highest of all illicit drugs in the United States. Human studies have demonstrated that a significant subset of chronic cannabis users have difficulty quitting cannabis use and consistently exhibit a cluster of withdrawal symptoms after abrupt cessation of cannabis use. Such symptoms include disturbances in sleep and affect, e.g., irritability, restlessness, anxiety, and dysphoria or depression. Many chronic cannabis users report an average of 6.4 withdrawal symptoms of at least moderate severity, a number that exceeds the criteria set by American Psychiatric Association for substance-withdrawal disorders (i.e., 2-4). However, there are currently no accepted pharmacotherapies for the management of cannabis withdrawal. Existing treatments are all of limited efficacy and do not address undesirable consequences of early abstinence from cannabis, e.g., negative affect and sleep disturbance that may prompt relapse.

There is a need for better means for preventing relapse to alcohol dependence and for treating symptoms or disorders associated with protracted abstinence. There is also a need in the art for effective treatment for cannabis dependence and withdrawal. The present invention addresses these and other needs.

### SUMMARY OF THE INVENTION

In one aspect, the invention provides methods for treating alcohol dependence and preventing relapse. The methods involve administering to a subject following acute alcohol withdrawal or in protracted alcohol abstinence a therapeutically effective amount of gabapentin, or a pharmaceutically acceptable salt thereof. Some of these methods are intended to treat alcohol dependence and prevent relapse in subjects who have undergone acute alcohol withdrawal. In some methods, the subjects to be treated are those who have been in abstinence from alcohol for at least about 1-5 days, 5-10 days, 10-45 days, 45-120, 120-180 days or longer. In some methods, the subjects to be treated are suffering from symptoms of protracted abstinence, e.g., disturbance in sleep and affect. Typically, subjects are treated for a period that is at least 1 week, 1 month, 3 months, 6 months, 1 year, and 2 years or longer. In some methods, the subjects to be treated are administered with a daily gabapentin dosage of between about 20 to about 10,000 mg. Preferably, the subjects are administered with a daily gabapentin dosage of between about 100 mg to about 5000 mg, and more preferably, with a daily gabapentin dosage of between about 300 mg or 500 mg to about 2000 mg.

In a related aspect, the invention provides methods for reducing alcohol consumption or craving by a subject with alcohol dependence. These methods entail administering to a subject following acute alcohol withdrawal or in protracted abstinence a therapeutically effective amount of gabapentin, or a pharmaceutically acceptable salt thereof. Typically, subjects suitable for these methods have undergone acute alcohol withdrawal. For example, the subjects can be those who have been in abstinence from alcohol for at least about 1-5 days, 5-10 days, 10-45 days, 45-120, 120-180 days or longer. Some of these methods are intended for subjects who suffer from symptoms of protracted abstinence, e.g., disturbance in sleep and affect. To help or facilitate the subjects to refrain from or reduce alcohol consumption, the subjects can be administered with gabapentin for a period that is at least 1 week, 1 month, 3 months, 6 months, 1 year, 2 years or longer. Typically, the subjects are administered with gabapentin at a daily gabapentin dosage of between about 20 to about 10,000 mg. A preferred daily dosage is between about 100 mg to about 5000 mg. In some more preferred embodiments, the subjects are administered a daily dosage of between about 300 mg or 500 mg to about 2000 mg, and most preferably between about 900 mg to about 1800 mg.

In another aspect, the invention provides methods for treating or alleviating symptoms of protracted alcohol abstinence. Such methods involve administering to a subject following acute alcohol withdrawal or in protracted alcohol abstinence a therapeutically effective amount of gabapentin. Typically, the subjects to be treated with these methods have undergone acute alcohol withdrawal. Examples of symptoms that can be treated with these methods include disturbance in sleep and affect. Often, the subjects to be treated have been in abstinence from alcohol for at least about 1-5 days, 5-10 days, 10-45 days, 45-120, 120-180 days or longer. The subjects can be treated for a period that is at least 1 week, 1 month, 3 months, 6 months, 1 year, and 2 years or longer. The typical daily dosage of gabapentin the subjects receive is in the range of between about 20 to about 10,000 mg. A preferred daily dosage range is between about 100 mg to about 5000 mg. In some preferred embodiments, the subjects are administered with a daily gabapentin dosage of between about 300 mg or 500 mg to about 2000 mg.

In another aspect, the invention provides methods for treating cannabis dependence. These methods involve administering to a subject with cannabis dependence a therapeutically effective amount of gabapentin, or a pharmaceutically acceptable salt thereof. Some of these methods are intended to treat cannabis dependence in subjects who are currently using cannabis. Some of the methods are intended to help or enable the subjects to cease or reduce cannabis use. Some other methods are directed to treating subjects who have ceased or discontinued cannabis use. For example, the methods can be employed to treat or alleviate symptoms of acute cannabis withdrawal. The methods can also be used to treat subject who are in protracted abstinence from cannabis use. The subjects can be treated with gabapentin to alleviate symptoms associated with protracted cannabis withdrawal, e.g., disturbance in sleep or affect. Some of these methods are intended to prevent relapse in subjects who have ceased or discontinued cannabis use. Typically, the subjects can be treated with gabapentin for a period that is at least 1 week, 1 month, 3 months, 6 months, 1 year, and 2 years or longer. The daily dosage of gabapentin to be administered to the subjects is usually in the range of between about 100 to about 5000 mg. Preferably, the subjects are administered with a daily gabapentin dosage of between about 300 mg or 500 mg to about 2000 mg.

A further understanding of the nature and advantages of the present invention may be realized by reference to the remaining portions of the specification and claims.

### BRIEF DESCRIPTION OF THE DRAWINGS

FIG. 1 illustrates effects of gabapentin vs. placebo on alcohol and affective cue-induced craving for alcohol in recently abstinent individuals with alcohol dependence (alcoholics).

FIG. 2 illustrates effects of gabapentin vs. placebo on affective cue-induced arousal in recently abstinent individuals with alcohol dependence (alcoholics).

FIG. 3 illustrates that gabapentin was associated with significantly greater reductions in THC/Cr (t=−1.42, p<0.02) and g/wk (t=−2.68, p<0.01) than placebo in individuals with cannabis dependence. Shown in the figure are grams of marijuana used per week and corresponding THC/Cr for Week-1 through Week 12. Inset graph is an enlargement of the last 3 study weeks i.e. Week 10-12.

FIG. 4 illustrates that gabapentin significantly reduced sleep disturbance (t=−2.21, p<0.02) and daytime dysfunction (t=−2.26, p<0.025) relative to placebo in individuals with cannabis dependence. Shown in the figure are Pittsburgh Sleep Quality Index components: sleep disturbance and daytime dysfunction for Week-1 through Week 8.

FIG. 5 illustrates that gabapentin was associated with significantly greater decreases in Marijuana Withdrawal Checklist (MWC) total score (t=−2.00, p<0.02), nervousness (t=5.33, p<0.001), and difficulty concentrating (t=2.6, p<0.02), with a trend for less fatigue (t=−1.10, n.s.) than placebo in individuals with cannabis dependence. Shown in the figure are MWC total score and individual items measuring fatigue, nervousness and difficulty concentrating.

FIG. 6 illustrates that mood improved (t=−1.96, p<0.05) and craving decreased significantly more (t=−2.63, p<0.01) with gabapentin than placebo in patients with marijuana dependence. Shown in the figure are mood and craving means pre and post-randomization comparing gabapentin to placebo.

FIG. 7 shows that administration with gabapentin reduces alcohol consumption in subjects after acute withdrawal.

FIG. 8 shows that gabapentin treatment attenuated daytime dysfunction in subjects in protracted abstinence.

FIG. 9 shows effect of gabapentin treatment on sleep quality in subjects in protracted alcohol abstinence.

FIG. 10 shows that gabapentin treatment attenuated negative affective symptoms in subjects in protracted alcohol abstinence.

FIG. 12 shows that subjects receiving gabapentin treatment displayed an increased number of abstinent days per week (FIG. 11).

FIG. 12 shows that gabapentin treatment increased rate of complete abstinence.

FIG. 13 shows that gabapentin treated subjects have a decreased number of heavy drinking days (FIG. 13) during the treatment period.

FIG. 14 shows that gabapentin treatment resulted in an increased rate of no heavy drinking over the whole treatment period.

FIG. 15 shows that treatment effect of gabapentin was sustained for up to 12 weeks after completing treatment.

FIG. 16 shows that gabapentin treated subjects have a decreased craving (FIG. 16).

FIG. 17 shows that gabapentin treated subjects have a decreased drinking frequency.

FIG. 18 shows that gabapentin treated subjects have a decreased drinking quantity.

FIG. 19 shows that gabapentin treated subjects have an improved liver functioning.

FIG. 20 shows that gabapentin treatment leads to a decrease in overall severity of alcoholism.

FIG. 21 shows that subjects receiving gabapentin have a reduced cigarette smoking.

### DETAILED DESCRIPTION OF THE INVENTION

### I. Overview

The present invention relates to methods for treating subjects with substance dependence, especially alcohol dependence and/or cannabis dependence. The present inventors discovered that gabapentin (Neurontin®) is effective for reducing alcohol craving in subjects with alcohol dependence (e.g., subjects with protracted alcohol abstinence) or cannabis use in subjects with cannabis dependence. The inventors also found that gabapentin is effective for treating or ameliorating acute and/or protracted withdrawal symptoms associated with alcohol or cannabis dependence, e.g., craving, disturbances in sleep and negative affective symptoms (e.g., symptoms of depression, anxiety, irritability, anger and etc.).

Specifically, as detailed in Example 1, the inventors investigated effects of gabapentin on symptoms of protracted abstinence in alcohol-dependent individuals who were exposed to alcohol cues (sight and smell of the subject's favorite alcoholic beverage) without consumption. The inventors found a significant attenuating effect of gabapentin (vs. placebo) on several measures of subjective craving for alcohol as well as for effectively-evoked craving. Similarly, gabapentin was also found to significantly improve several measures of sleep quality in these individuals. In addition, the results from these studies indicate that side effects of gabapentin were minimal. Taken together, these studies indicated suggest that gabapentin can be effective for treating the protracted abstinence phase in alcohol dependence. These results also suggest that gabapentin can be employed to reduce alcohol consumption in subjects with alcohol dependence, and to alleviate various symptoms associated with alcohol withdrawal.

In addition, the inventors examined effects of gabapentin treatment on subjects with cannabis dependence (Examples 2 and 3). It was found that administration of gabapentin with a daily dosage of 1200 mg resulted in reduced cannabis use and improved symptoms associated with protracted cannabis withdrawal. Gabapentin was also found to attenuate withdrawal symptoms, both during the early weeks of the trial when such symptoms would be expected to peak and during later weeks when protracted symptoms involving mood, craving and sleep were still present. Moreover, gabapentin was found to be safe and well tolerated, with high rates of medication compliance.

Gabapentin received FDA approval in 1993 for use an adjunctive agent for the treatment of complex partial seizures with and without generalization, and is used off label for treatment of a variety of psychiatric disorders. Clinical reports and controlled trials have supported the efficacy of gabapentin for treatment of symptoms associated with acute withdrawal from alcohol. However, prior to the subject invention, it was not known or expected that the drug would also be effective for treating symptoms in subjects with protracted abstinence and preventing relapse. In addition, there has been no reported or suggested use of gabapentin in the treatment of cannabis dependence.

In accordance with the novel discoveries by the present inventors, the present invention provides methods of using gabapentin in the treatment of conditions or symptoms that are induced by or associated with cannabis withdrawal (e.g., cessation of cannabis use) or protracted alcohol withdrawal. The invention also provides methods of using gabapentin as a means to help or enable subjects with cannabis dependence to reduce cannabis use, and to alleviate other symptoms associated with cannabis withdrawal (e.g., negative affect, craving and sleep disturbance). Also provided in the invention are methods using gabapentin in subjects who have undergone acute alcohol withdrawal to reduce alcohol consumption or craving during protracted abstinence. The invention further provides methods of using gabapentin for preventing relapse into alcohol or cannabis dependence following cessation or discontinuation of substance abuse or consumption. The following sections provide more detailed guidance for practicing the methods of the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Diagnostic and Statistical Manual of Mental Disorders, American Psychiatric Publishing, Inc., 4^{th} Ed. (1994; “DSM-IV”) and (2000; “DSM-IV-TR”); Academic Press Dictionary of Science and Technology, Morris (Ed.), Academic Press (1^{st} ed., 1992); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “analog” is used herein to refer to a molecule that structurally resembles a reference molecule (e.g., gabapentin) but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

As used herein, a gabapentin compound refers to the generic term for the chemical compound (1-aminomethyl)-1-cyclohexaneacetic acid (gabapentin), as well as its various salts or analogs known in the art to possess the same or similar pharmaceutical properties. For example, pregabalin (Lyrica®) is considered an analog of gabapentin and is suitable for the practice of the present invention.

Cannabis is a generic term used to denote the several psychoactive preparations of the marijuana (hemp) plant, Cannabis sativa. They include marijuana leaf (in street jargon: grass, pot, dope, weed, or reefers), bhang, ganja, or hashish (derived from the resin of the flowering heads of the plant), and hashish oil. Cannabis contains at least 60 cannabinoids, several of which are biologically active. The most active constituent is Δ⁹-tetrahydrocannabinol (THC). THC and its metabolites can be detected in urine for several weeks after usage of cannabis (usually by smoking).

The terms “treatment” or “treating” as used herein refers to partially or completely alleviating, inhibiting, preventing, ameliorating and/or relieving a disease or disorder, or one or more symptoms thereof. Treatment can be therapeutic or prophylactic in nature.

The term “subject” or “patient” refers to a mammal, e.g., human or non-human animals. In particular, the term refers to a male or female human being of any race, national origin, age, physiological make-up, genetic make-up, disease predisposition, height, or weight. Unless otherwise noted, the term subject as used in the present disclosure typically refers to one who has dependence on a psychoactive substance (e.g., alcohol or cannabis), is suspected to have substance dependence or is at risk of developing substance dependence.

The terms “suffer” or “suffering” as used herein refers to one or more conditions that a subject has been diagnosed with, is at risk of developing, or is suspected to have.

Blood alcohol level (BAL) refers to the concentration of alcohol (ethanol) present in blood. It is usually expressed as mass per unit volume, but different countries may express it differently or use different units; examples include milligrams per 100 milliliters (mg/100 ml or, incorrectly, mg percent), milligrams per liter (mg/l), grams per 100 milliliters (g/100 ml), grams percent, and millimoles per liter. A concentration of 8 parts per thousand would be expressed in legal terminology in USA as 0.08% or 80 mg/100 ml in some other countries.

A psychoactive or psychotropic substance (e.g., alcohol or cannabis) is a chemical substance that acts primarily upon the central nervous system where it alters brain function, resulting in temporary changes in perception, mood, consciousness and behavior. Psychoactive substances affect the brain and bring about subjective changes in mood that the user may find pleasant. Many psychoactive substances are abused, that is, used outside of the guidance of a medical professional and for reasons other than their original purpose. With sustained use, physical dependence may develop, making the cycle of abuse even more difficult to interrupt.

The term craving refers to very strong desire for a psychoactive substance (e.g., alcohol or cannabis) or for the intoxicating effects of that substance. Craving is a term in popular use for the mechanism presumed to underlie impaired control and to correspond to drinking relapse: it is thought by some to develop, at least partly, as a result of conditioned associations that evoke conditioned withdrawal responses. Craving may also be induced by the provocation of any physiological arousal state resembling an alcohol or drug withdrawal syndrome.

As used herein, the term substance abuse is used to refer to non-medical or unsanctioned use of psychoactive substances (e.g., alcohol or cannabis). Typically, substance abuse is indicated by continued use of a psychoactive substance despite knowledge of having a persistent or recurrent social, occupational, psychological or physical problem that is caused or exacerbated by the use. Unless otherwise noted, the term substance abuse is intended to include psychological dependence, physical dependence, tolerance, a maladaptive pattern of alcohol use, preoccupation with a psychoactive substance such as alcohol or cannabis, and/or the prevalence of withdrawal symptoms upon cessation of use.

The term addiction refers to repeated use of a psychoactive substance or substances (e.g., alcohol and cannabis), to the extent that the subject is periodically or chronically intoxicated, shows a compulsion to take the preferred substance (or substances), has great difficulty in voluntarily ceasing or modifying substance use, and exhibits determination to obtain psychoactive substances by almost any means. Typically, tolerance is prominent and a withdrawal syndrome frequently occurs when substance use is interrupted. The life of the addict may be dominated by substance use to the virtual exclusion of all other activities and responsibilities. The term addiction also conveys the sense that such substance use has a detrimental effect on society, as well as on the individual; when applied to the use of alcohol, it is equivalent to alcoholism. Unless otherwise noted, the term addiction is used interchangeably with dependence or dependence syndrome.

Dependence or dependence syndrome refers to a cluster of behavioral, cognitive, and physiological phenomena that may develop after repeated substance use (e.g., alcohol or cannabis use). Typically, these phenomena include a strong desire to take the drug, impaired control over its use, persistent use despite harmful consequences, a higher priority given to drug use than to other activities and obligations, increased tolerance, and a physical withdrawal reaction when drug use is discontinued. In International Statistical Classification of Diseases and Related Health Problems (ICD-10), the diagnosis of dependence syndrome is made if three or more of six specified criteria were experienced within a year. The dependence syndrome may relate to a specific substance (e.g., alcohol or other controlled substance), a class of substances, or a wider range of pharmacologically different substances.

Alcohol abuse becomes alcohol dependence when drinkers begin to experience a craving for alcohol, a loss of control of their drinking, withdrawal symptoms when they are not drinking and an increased tolerance to alcohol so that they have to drink more to achieve the same effect. Alcohol dependence is a chronic and often progressive disease that includes a strong need to drink despite repeated problems.

The term detoxification refers to the process by which a subject is withdrawn from a psychoactive substance (e.g., alcohol). As a clinical procedure, the withdrawal process is carried out in a safe and effective manner, such that withdrawal symptoms are minimized. Typically, the subject is clinically intoxicated or already in withdrawal at the outset of detoxification. Detoxification may or may not involve the administration of medication. When it does, the medication given is usually a drug that shows cross-tolerance and cross-dependence to the substance taken by the patient. The dose is calculated to relieve the withdrawal syndrome without inducing intoxication, and is gradually tapered off as the patient recovers. Detoxification as a clinical procedure implies that the individual is supervised until recovery from intoxication or from the physical withdrawal syndrome is complete. The term “self-detoxification” is sometimes used to denote unassisted recovery from intoxication or withdrawal symptoms.

Cessation or discontinuation of alcohol consumption (or cannabis use) means a complete abstinence from consuming any alcoholic beverage (or cannabis related substance such as cannabinoids or marijuana). A substantial reduction of alcohol consumption (or cannabis use) means that the amount of alcohol (or cannabis) being consumed on a daily or weekly basis is reduced by at least 50%, 75% or 90% relative to the amount that causes alcohol dependence (or cannabis dependence) in a subject or the amount the subject has been using to maintain dependence.

The term withdrawal, withdrawal symptoms, or withdrawal syndrome refers to a collection of symptoms of variable clustering and degree of severity which occur on cessation or abrupt reduction of use of a psychoactive substance (e.g., alcohol or cannabis) that has been taken repeatedly, usually for a prolonged period and/or in high doses. The syndrome may be accompanied by signs of physiological disturbance. A withdrawal syndrome is one of the indicators of a dependence syndrome. It is also the defining characteristic of the narrower psycho-pharmacological meaning of dependence. The onset and course of the withdrawal syndrome are time-limited and are related to the type of substance and dose being taken immediately before cessation or reduction of use. Typically, the features of a withdrawal syndrome are the opposite of those of acute intoxication. Unless otherwise noted, the term withdrawal or withdrawal syndrome refer to acute withdrawal or acute withdrawal syndrome. As noted below, it should be distinguished from protracted withdrawal and symptoms thereof.

For short-acting substance such as alcohol, the most severe signs and symptoms of withdrawal (i.e., acute withdrawal) usually begin within hours of the subject's last use. They usually do not last more than 2 days. With a long-acting substance, withdrawal symptoms may not begin for several days and usually reach peak intensity after 5 to 10 days. The most severe drug-withdrawal symptoms, during the initial stages of detoxification, constitute the acute abstinence syndrome. The adjective “acute” distinguishes the syndrome from a “chronic” or protracted withdrawal syndrome (or protracted abstinence syndrome), in which signs and symptoms of abstinence from a psychoactive substance may continue for weeks to months after cessation of use.

As described herein for alcohol and cannabis, the rough time periods corresponding to acute withdrawal and protracted withdrawal for various psychoactive substances are all known in the art. However, it should be noted that the exact line between the period of acute withdrawal and the period corresponding to protracted withdrawal can vary, depending on the specific subject to be treated and the specific substance used by the subject. As described herein (e.g., the Examples below), the skilled artisan can readily determine whether a subject is in acute abstinence or protracted abstinence based on the history of substance use, timeline of detoxification and cessation, and any accompanying withdrawal syndromes.

Acute alcohol withdrawal or acute alcohol withdrawal syndrome refers to a cluster of symptoms that may occur when a subject with alcohol abuse or alcohol dependence acutely ceases or substantially reduces alcohol consumption. Such symptoms include, e.g., tremor, sweating, anxiety, agitation, depression, nausea, and malaise. The syndrome usually occurs during a withdrawal period which starts about 5-48 hours after cessation or reduction of alcohol consumption and, when uncomplicated, typically abates after 2-5 days. It may be complicated by grand mal seizures and may progress to delirium (known as delirium tremens).

Acute cannabis withdrawal refers to a cluster of symptoms after abrupt cessation or substantial reduction of cannabis use by a subject. These symptoms can include, e.g., disturbed sleep, symptoms of depression, irritability, restlessness and anxiety. Marijuana withdrawal appears less severe than alcohol withdrawal because it produces less dramatic physical symptoms. This could be, in part, a function of an elimination half-life of 7 days, with up to 30 days for complete elimination that result in a more protracted withdrawal process (Ashton, Br. J. Psychiatry 178:101-6, 2001).

Protracted withdrawal or protracted withdrawal (or abstinence) syndrome refers to the occurrence of symptoms of a withdrawal syndrome, usually minor but nonetheless discomforting, for several weeks or months after the acute physical withdrawal syndrome has abated, and that may motivate a return to substance use. For subjects with alcohol dependence, symptoms of protracted withdrawal include, e.g., craving for alcohol, sleep disturbance (insomnia), anxiety, depression, and irritability. Symptoms of protracted cannabis withdrawal include, e.g., disturbed sleep, depression, aggression/anger, nervousness/anxiety, craving for marijuana, decreased appetite/weight loss, irritability and restlessness. Symptoms may be precipitated or exacerbated by the sight of alcohol or the drug of dependence (e.g., cannabis), or by return to the environment previously associated with alcohol or other drug use.

The term “abstinence” refers to refraining from psychoactive substance use (e.g., alcohol or cannabis), whether as a matter of principle or for other reasons. This term should not be confused with “abstinence syndrome” or “withdrawal syndrome”. Unless otherwise noted, acute abstinence refers to refraining from substance use during the period immediately following detoxification and cessation of substance use (i.e., acute abstinence period). During acute abstinence period, the subject typically undergoes or experiences acute withdrawal syndrome. Unless otherwise noted, protracted abstinence refers to refraining from substance after the acute abstinence period. Thus, for alcohol abstinence, protracted abstinence refers to refraining from alcohol consumption after the acute withdrawal period, e.g., at least about 1 day, 2 days, 3 days, 5 days, 1 week, 2 weeks, 1 month, 3 months, 1 year or even longer after cessation or substantial reduction of alcohol consumption.

The term “negative affect” or “disturbance in affect” refers to a cluster of negative affective symptoms, e.g., symptoms of depression, anxiety, irritability, anger and etc.

As used herein, relapse is a return to alcohol drinking or other drug use (e.g., cannabis use) after a period of abstinence, often accompanied by reinstatement of dependence symptoms. Relapse prevention refers to therapeutic procedures employed in case of alcohol or other drug problems to help individuals avoid or cope with lapses or relapses to uncontrolled substance use. The procedures may be used with treatment based on either moderation or abstinence, and in conjunction with other therapeutic approaches.

### III. Treatment Schemes and General Considerations

The invention provides methods for treating subjects suffering from substance dependence especially alcohol dependence or cannabis dependence, e.g., treating disorders or alleviating symptoms associated with or induced by substance dependence (e.g., acute or protracted withdrawal syndrome). These methods entail the administration of a therapeutically effective amount of gabapentin or a salt or analog thereof (e.g., pregabalin). Any subjects who are suffering from substance abuse or are at risk of developing or relapsing into substance abuse are suitable for treatment with methods of the present invention. In some preferred embodiments, subjects suitable for treatment with methods of the invention are those that will meet DSM-IV criteria for current alcohol dependence or cannabis dependence. Dependence syndromes and withdrawal syndromes associated with discontinuation of the use of a psychoactive substance (e.g., alcohol or cannabis) are defined and described in detail herein or in DSM-IV.

Some methods of the invention are directed to treating alcohol dependence and preventing relapse in subjects who have previously developed alcohol dependence and are currently at risk of relapsing to alcohol dependence. Typically, the subjects have undergone the acute withdrawal period or acute abstinence. Therefore, some methods of the invention are directed to treating subjects who have gone through the acute alcohol withdrawal period and/or are in protracted abstinence. Subjects to be treated with these methods typically have ceased or substantially reduced alcohol consumption for at least about 1-5 days, e.g., at least 1 day, at least 2 days, at least 3 days, at least 4 days or at least 5 days. Some other methods are directed to treating subjects who have ceased or substantially reduced alcohol consumption for a period that is at least 7 days, 2 weeks, 1 month, 3 months, 6 months, 1 year, 2 years or longer.

Some methods of the invention are intended to treat subjects with cannabis dependence and who are currently using cannabis. These subjects are administered with gabapentin to treat dependence (e.g., to reduce or cease cannabis use) and to alleviate symptoms associated with acute withdrawal. Some other methods are directed to treating subjects who have ceased or reduced cannabis consumption. These subjects are administered with gabapentin to treat or alleviate symptoms associated with protracted withdrawal and also to prevent relapse.

The Examples below described exemplary procedures for carrying out the treatment in accordance with the present invention. For example, assessment of severity of substance dependence of a subject can be performed at the beginning of the treatment period and also monitored along the process. This can be accomplished with methods or measures well known in the art. For example, subjects with alcohol dependence or cannabis dependence can be screened according to the respective criteria set forth in DSM-IV or DSM-IV-TR. Alcohol dependence scale (ADS) provides a reliable and valid quantitative measure of severity of alcohol dependence. See, e.g., Skinner & Horn, Alcohol Dependence Scale: Users Guide, Toronto, Canada, Addiction Research Foundation, 1984. In addition, breath alcohol concentrations (BAC) can be obtained at every treatment visit to confirm self report of abstinence. Subjects undergoing treatment for alcohol dependence may also be monitored by measuring blood alcohol level (BAL). The BAL is often extrapolated from measurements made on breath or urine or other biological fluids in which the alcohol concentration bears a known relationship to that in the blood. The Widmark calculation is a technique for estimating BAL at a given time after alcohol ingestion by extrapolating from BALs at known times.

Similar methods are also available for assessing cannabis dependence and related characteristics. For example, the criteria set forth in DSM-IV or DSM-IV-TR can be employed to identify subjects with cannabis dependence and severity of symptoms associated with acute or protracted cannabis withdrawal. Other methods that may be used to examine cannabis dependence include the Fagerstrom test for nicotine dependence (FIND) (Heatherton et al., Br. J. Addict. 86:1119-27, 1991). FIND is a 6-item rating scale of nicotine dependence and can be employed to assess cannabis dependence in subjects. Illicit drug use index (IDUI) (Clayton and Voss, DHHS Pub. No. (ADM) 81-1167, 1981; and NIDA Res Monogr. 39:1-187, 1981) allows assessment of the frequency and duration of illicit drug use. Other methods and measures for assessing cannabis dependence are described in the Examples below.

Administration of gabapentin to subjects with alcohol dependence and/or cannabis dependence can be performed in accordance with procedures described herein (e.g., as exemplified in the Examples) or otherwise well known in the art. Each administration usually employs a pharmaceutically effective dosage of a gabapentin compound or a pharmaceutical composition comprising the gabapentin compound. Typically, the effective dosage would be in the range of about 50 mg to about 10,000 mg per day. For example, some methods entail administration to subjects in need of treatment with a gabapentin dosage of about 100 mg, 250 mg, 300 mg, 500 mg, 1000 mg, 2000 mg or 5000 mg per day. In some preferred embodiments, as exemplified in the Examples below, subjects are administered with a dosage of between about 900 mg to about 1800 mg per day. For example, some embodiments employ a daily dosage of 900 mg to treat alcohol dependence. In some other embodiments, the dosage administered is about 1800 mg day for treating alcohol dependence. In some other preferred embodiments, subjects with cannabis dependence are administered with a daily dosage of about 1200 mg day.

In some embodiments, as exemplified in the Examples, subjects can receive a gradually increasing daily dosage during one stage (e.g., at the beginning) of the treatment period and/or decreasing daily dosage during another stage (e.g., towards the end) of the treatment period). In some other embodiments, subjects are administered with gabapentin in a slow release or sustained release formulation. In these embodiments, the dosage and frequency of administration can be adjusted in accordance with pharmaceutical practice well known in the art. Also, otherwise than the daily dosages noted above, the amount of gabapentin administered to the subjects may also be measured according to their body weight. For example, the dosage can be between about 0.1 and 500 mg/kg body weight of the subject to be treated. More preferably, the effective dosage is between about 0.5-250, 1-100 or 5-40 mg/kg body weight.

Typically, the subjects in need of treatment can be administered with gabapentin for a treatment period that can last at least a week, two weeks, a month, two months, six months, 1 year, 2 years or longer. Preferably, the treatment period lasts at least one month, two months or longer. In some embodiments, subjects are administered with the medication on daily basis. The subject can be administered with the compound once a day or several times a day. In some preferred embodiments, as exemplified in the Examples below, subjects are administered with the medication three times a day. In some other embodiments, the medication may be administered to subjects in need of treatment once, twice, four times or more times a day.

Other than daily administration, the medication can also be administered to the subjects based on other schedules throughout the treatment period. These include administration of the medication at intervals of, e.g., bi-daily, every three days, weekly or longer. Intervals can also be irregular as indicated by measuring blood level of administered gabapentin compound or the severity of symptoms to be treated or alleviated. For example, subjects can be administered with the medication on a more frequent schedule (e.g., once, twice or more times a day) at the beginning of the treatment period. As the symptoms improve during the treatment period, the subjects can then be administered with the drug on a less frequent schedule (e.g., once daily, bi-daily, or weekly). In addition, if blood level of the administered compound increases over the treatment period, the medication can also be accordingly administered less frequently.

In some embodiments, the gabapentin compound can be administered as a sustained release formulation, in which case less frequent administration is required. The dosage and frequency of administration can vary depending on whether the treatment is prophylactic or therapeutic. In prophylactic applications, a relatively low dosage is administered at relatively infrequent intervals over a long period of time. If necessary, some subjects may continue to receive treatment for the rest of their lives. In therapeutic applications, a relatively high dosage at relatively short intervals is sometimes required until progression of the symptoms is reduced or terminated, and preferably until the subject shows partial or complete amelioration of symptoms to be treated. Thereafter, the subject can be administered a prophylactic regime.

In some methods, the subjects are administered with a gabapentin compound in conjunction with manual-guided behavioral counseling. Such behavioral counseling is described in more detailed in the Examples below. For example, the subjects can receive counseling at intervals (e.g., weekly) throughout the treatment period. In addition, to ensure long term effect of treatment, subjects can also receive counseling and assessments once the treatment period has ended, e.g., once every 4 weeks, 8 weeks, or 12 weeks post-treatment.

For the purposes of the present application, treatment can be monitored by assessing the amount of alcohol or cannabis consumption and/or observing one or more of the improved symptoms associated with cannabis withdrawal or protracted alcohol withdrawal, e.g., disturbance in sleep or affect. Methods for assessing substance use by a subject, the primary outcome of treatment, are well known in the art. Monitoring procedures for improved symptoms are also known in the art, e.g., as described in more detail in the Examples below. For example, to assess the primary outcome of treatment of alcohol dependence, the method of timeline followback interview (TLFB) can be used. TLFB provides quantity and frequency estimates of alcohol intake at baseline and throughout the study using a standard drink format. It was described by Sobell and Sobell, Timeline Follow-back: A technique for assessing self-reported alcohol consumption, In: Litten, R. Z., and Allen, J. P., eds. Measuring Alcohol Consumption: Psychosocial and Biological Methods, Totowa, N.J., Humana Press, 1992. TLFB provides a calendar of 1-3 months that lists visual cues to aid persons in retrospective recall of behavior. It should be noted that that no one method has been found to reliably and validly detect drinking that occurs between study visits. Therefore, TLFB data can be supported by other measures, e.g., weekly breathalyzers and monthly collateral informant reports and GGT.

For assessing primary outcome of treatment of cannabis withdrawal, Marijuana Withdrawal Discomfort Scale (WDS) (Budney et al., Arch. Gen. Psychiatry 58:917-24, 2001) can be employed. WDS allows assessment of severity for each of the 10 most frequently reported marijuana withdrawal symptoms: anger, craving, depression, decreased appetite, headaches, irritability, nervousness, restlessness, sleep difficulty, and strange dreams. Timeline followback interview (TLFB) can also be used to assess the pattern and frequency of cannabis smoking at baseline and throughout the treatment period (see, e.g., Fals-Stewart et al., J. Consult. Clin. Psychol. 68:134-44, 2000). In addition, urine THC/creatinine ratio provides a highly sensitive and specific quantitative analytic procedure to determine new marijuana use or abstinence (see, e.g., Huestis and Cone, Ther. Drug. Monit. 20:570-6, 1998). Other useful measures for assessing cannabis dependence and treatment are described in the Examples below.

Other than assessing the primary outcome of treatment, secondary outcome can also be analyzed during the treatment period with a number of means. For example, current drinking urges, difficulty resisting urges and anticipation of positive outcome or relief from negative affective state by drinking can be examined with the aid of the alcohol craving questionnaire-short form (ACQ-SF). This form was developed by Singleton et al. as described in Proceeding of the 56th Annual Meeting, The College on Problems of Drug Dependence, Inc., Volume II: Abstracts. NIDA Research Monograph 153. Rockville, Md., National Institute on Drug Abuse, page 289. Beck depression inventory (BDI) is a self-rating of severity of depressive symptoms. It was first published by Beck et al., Arch Gen Psychiatry 4: 561-571, 1961. Alcohol abstinence or relapse can be assessed by examining serum gamma-glutamyl transferase (a biochemical marker), as described Kristenson and Trell, Br J Addict. 77:297-304, 1982. Subjective sleep quality and disturbance can be assessed with the aid of Pittsburgh sleep quality index (PSQI). PSQI was described in, e.g., Buysse et al., Psychiatry Res. 28:193-213, 1989.

Secondary outcome of treatment of cannabis dependence can also be assessed with methods and measures well known in the art. For example, as with subjects with alcohol dependence, sleep in subjects with cannabis dependence can be evaluated with Pittsburgh sleep quality index (PSQI). Consequences can be examined with the marijuana consequences checklist (Budney et al., Addiction 93:493-503, 1998) which assesses the incidence of 26 frequently reported consequences of marijuana dependence, e.g., interpersonal problems, memory problems, and financial. Craving can be examined with the marijuana craving questionnaire (Budney et al., Arch. Gen. Psychiatry 58:917-24, 2001) which is a 10-item questionnaire that yields a total craving score and two subscale scores. Depressive severity can be analyzed with Beck depression inventory (BDI-II) (Beck et al., J. Pers. Assess. 67:588-97, 1996) which is a self-rating of severity of depressive symptoms (baseline and every study visit, 5 minutes). In addition, positive and negative affect schedule (PANAS) provides a quick reliable and valid measure of current positive and negative affect (Watson et al., J. Pers. Soc. Psychol. 54:1063-70, 1988). These methods as well as some other measures for assessing cannabis dependence are described in more detail in the Examples below.

### IV. Gabapentin Compounds and Other Therapeutic Agents

The methods of the invention employ a gabapentin compound as the active ingredient in the treatment of substance dependence. In some methods, the compound used is gabapentin which has the chemical name of compound (1-aminomethyl)-1-cyclohexaneacetic acid. In some other methods, a pharmaceutically acceptable gabapentin salt or analog compound with similar biological and/or pharmaceutical properties (e.g., pregabalin) is employed. For example, gabapentin salts suitable for the present invention include acid salt (e.g., gabapentin hydrochloride hydrate) and sodium salt (e.g., gabapentin hydrate). See, e.g., U.S. Pat. Nos. 4,024,175 and 4,087,544.

Gabapentin compounds can be readily obtained from commercial suppliers or synthesized de novo. For example, gabapentin is commercially marketed under the trade name of Neurontin® by Pfizer (New York, N.Y.). Chemical synthesis of gabapentin compound and its various salt forms or analogs (e.g., pregabalin) can be performed as described in, e.g., U.S. Pat. Nos. 4,024,175; 4,087,544; 4,894,476; 4,960,931; 6,255,526; 6,528,682; 6,576,790; 6,891,059. These patents describe various processes for the preparation of gabapentin and related salts or analogs from a variety of starting materials. For example, U.S. Pat. No. 4,024,175 described at least three methods of preparing gabapentin from cyclohexyl-1,1-diacetic acid. Each of these methods results in the formation of gabapentin hydrochloride salt, which may be converted to 1-(aminomethyl)-1-cyclohexaneacetic acid by treatment with a basic ion exchanger and then crystallized from a solvent such as ethanol/ether. U.S. Pat. No. 4,894,476 disclosed an improved method for converting the hydrochloride salt into the free amino acid. This involves pouring a deionized water solution of the salt over an ion exchange column, eluting with deionized water, producing a slurry from the eluate, adding an alcohol to the slurry, centrifuging and drying the slurry to obtain the free amino acid. U.S. Pat. No. 5,563,175 also disclosed methods of using GABA analogs to synthesize Gabapentin.

Alternative methods for preparing gabapentin have been described that do not proceed via the hydrochloride or any other mineral acid salt. Such methods include those described in U.S. Pat. Nos. 5,132,451, 5,095,148, 5,068,413. Each of these methods involves a cyanic intermediate which is hydrogenated under severe conditions to produce the free amino acid.

In some methods of the invention, a subject in need of treatment can be administered with a gabapentin compound alone. In some other methods, subjects are treated with gabapentin in combination with other treatment schemes or therapeutic agents for treating substance abuse. For example, for subjects suffering from alcohol dependence, the treatment can combine administration of gabapentin with other known drugs for treating alcohol dependence. Examples of the latter drugs include, e.g., disulfuram (Antabuse), naltrexone (Trexan®), acamprosate (Campral®), Nalmefene (Revex®), Fluoxetine (Prozac'), ondansetron (Zofran®) and Topiramate (Topamax®). For subjects with cannabis dependence, treatment with gabapentin can be supplemented with other pharmacological aids, e.g., buproprion or cannabinoid antagonist such as SR 141716A. In addition to drug intervention with gabapentin, subjects suffering from alcohol or cannabis dependence can also receive other treatments (e.g., acupuncture) or enroll in stress management activities (e.g., exercising, meditation or yoga). Stress management activities are helpful to maintaining long-term abstinence.

### V. Treating Alcohol Dependence and Preventing Relapse

The invention provides various methods for therapeutically or prophylactically treating symptoms associated with protracted alcohol abstinence. Typically, subjects to be treated are at least 2 days or 3 days after detoxification or cessation of alcohol consumption. More preferably, subjects receiving treatment are in protracted alcohol abstinence and have ceased or substantially reduced alcohol consumption at least 5 days ago. Protracted drug withdrawal or abstinence syndrome encompasses an aggregate of signs and symptoms that may continue for weeks or months after cessation of drug use. It is also termed “chronic” drug withdrawal or abstinence syndrome. In contrast, acute abstinence syndrome refers to the aggregate of withdrawal signs and symptoms that occur shortly after a person who is physically dependent on a drug stops taking it. When a substance abuser discontinues regular use, it takes weeks or months of abstinence after acute detoxification before the brain's pleasure mechanisms are able to achieve equilibrium through an increase in the production and utilization of neurotransmitters to levels that can maintain a stable and optimistic mood. Depending on the specific psychoactive substance, this period during which subjects experience distinct stages of physical and psychological symptoms can last from several months to more than a year. This period is the time of greatest relapse. For example, most subjects with alcohol dependence (as high as 98%) in the U.S. relapse within three months of detoxification.

Physiologically, the body changes during the protracted abstinence period. In the case of alcohol, changes in physiological activity are associated with sleep disturbance, lack of energy, transient attention deficit, impulsive and erratic behavior, and low frustration tolerance. Psychological and emotional signs and symptoms may include depression, anxiety, self-doubt and boredom, coupled with confusion, hostility, inappropriate coping responses and fear. Symptoms or disorders which accompany with protracted abstinence of alcohol consumption can often precipitate relapse. These include, e.g., negative mood, sleep disturbance, body aches, and craving. For example, anxiety is associated with subjects with protracted abstinence from alcohol.

Subjects at the various stages of protracted alcohol abstinence (i.e., following detoxification or cessation of alcohol consumption) as noted above can all be treated with the methods of the invention. For example, subjects to be treated can be in abstinence for at least 1 day, at least about 2-5 days, at least about 5-10 days, at least about 10-45 days, at least about 45-120 days, at least about 120-180 days or longer. Some methods of the invention are directed to enable or help the subjects who are in protracted abstinence to reduce or refrain from alcohol consumption. Some other methods of the invention are directed to preventing relapse in subjects in protracted alcohol abstinence. Still some other methods are directed to treating or alleviating symptoms associated with protracted alcohol withdrawal. As noted above, examples of such symptoms include disturbances in sleep and affect. All these methods involve administering to the subjects in need of treatment a pharmaceutical composition comprise an effective amount of a gabapentin compound or a pharmaceutically acceptable salt or analog thereof (e.g., pregabalin). Detailed steps and procedures for carrying out these methods are described above and further exemplified in the Examples below.

### VI. Treating Subjects with Cannabis Dependence and Preventing Relapse

The invention provides methods of using a gabapentin compound for treating subjects suffering from cannabis dependence. Cannabis encompasses a variety of substances prepared from the plant Cannabis sativa. In small doses, it is classified as a depressant. In larger doses, it can be a hallucinogen. The main substance in cannabis that causes the effects on the brain is a chemical called tetrahydrocannabinol (THC). The concentration of THC varies according to the part of the plant that is used, the variety of the plant and its growing conditions. Cannabinoids contained in cannabis (e.g., THC) are sometimes used therapeutically for glaucoma and to counteract nausea in cancer chemotherapy.

Acute marijuana use impairs cognitive development (capabilities for learning), including associative properties, recall of previously learned items, and psychomotor performance in a wide variety of tasks, such as motor coordination, divided attention and operative tasks of many types. Human performance on complex machinery can be impaired for as long as 24 hours after smoking as little as 20 mg of THC in cannabis, with an increased risk of motor vehicle accidents among persons who drive when intoxicated by cannabis. Chronic use of cannabis is associated with selective impairments in cognitive functioning which include the organization and integration of complex information involving various mechanisms of attention and memory processes. Long-term cannabis smoking is also associated with epithelial injury of the trachea and major bronchi, airway injury, lung inflammation, and impaired pulmonary defense against infection. Cannabis use during pregnancy is associated with impairment in fetal development leading to a reduction in birth weight and to postnatal risk of rare forms of cancer. Chronic users are at high risk for developing a dependence syndrome characterized by tolerance, impairment, and loss of control over use of the substance. Several hours per day may be spent acquiring and smoking marijuana, and cognitive and motivational handicaps are likely which may interfere with occupational performance, family, school or recreational activities.

Epidemiological studies have consistently found marijuana dependence to be by far the most prevalent of the illicit substance dependence disorders in this country, with more than 2 million Americans estimated to meet diagnostic criteria (2001 National Household Survey on Drug Abuse). Conversely, there is a paucity of treatment trials for cannabis dependence. Published, controlled treatment trials for marijuana dependence are few and have focused primarily on cognitive-behavioral techniques to prevent relapse. Treatment seekers averaged over 10 years of daily use and over 6 serious quit attempts. Relapse rates tended to be large and comparable to those for alcohol, tobacco and other drugs of abuse.

Symptoms of protracted abstinence provide predictors of relapse in cannabis dependence. They can occur after the subjects have gone through the acute abstinence period, e.g., about at least 1 day, 3 days, 6 days, 10 days, 14 days, 1 month, 2 months, 6 months, 1 year or longer after cessation of cannabis use. A DSM-IV criterion for substance dependence is the experience of withdrawal symptoms when trying to stop use. Pre-clinical and human laboratory studies of abstinence following THC/marijuana administration have identified a cluster of withdrawal symptoms that have been replicated in clinical samples with marijuana dependence. Symptoms reliably include aggression/anger, craving for marijuana, decreased appetite/weight loss, irritability, restlessness, sleep difficulty/strange dreams, nervousness/anxiety, and depression. A carefully controlled study of the time course of marijuana withdrawal symptoms found onset typically occurred between Days 1-3, peak effects between Days 2-6, and most effects lasted 4-14 days, although some, e.g., sleep disturbances, were unresolved at the conclusion of the 45 day study period (Budney et al., J. Abnorm. Psychol. 112:393-402, 2003).

In some embodiments, the invention provides methods to treat subjects who are suffering from cannabis dependence and/or are still using cannabis immediately prior to treatment. In order to help such subjects reduce or cease cannabis use, these subjects are administered with a pharmaceutical composition that comprises an effective amount of gabapentin compound or a pharmaceutically acceptable salt or analog thereof. In some related embodiments, the invention provides methods for treating or alleviating symptoms associated with acute cannabis withdrawal. These symptoms can occur within the acute withdrawal period which usually encompasses the few first hours to first few days, e.g., 1, 2, 5, 7, 10, 14, 30, 45 days or longer after cessation. In these embodiments, subjects with cannabis dependence but who have just ceased or discontinued cannabis use are administered with a gabapentin compound or a pharmaceutical composition comprising an effective amount of a gabapentin compound. Such treatment can eliminate or alleviate the various symptoms associated with cannabis withdrawal in the subject, e.g., irritability, sleep difficulties, restlessness, anxiety, and depression.

In some other embodiments, the invention provides methods for treating subjects who have undergone acute cannabis withdrawal and are in protracted cannabis abstinence. Subjects suitable for treatment with these methods have typically ceased cannabis use for a period that is, e.g., at least 5 days, 1 week, 2 weeks, 1 month, 2 moths, 6 months or longer. These methods involve administering to the subjects in need of treatment a pharmaceutical composition comprising an effective amount of a gabapentin compound or a pharmaceutically acceptable salt or analog thereof. Some of these methods are directed to preventing relapse in subjects during protracted abstinence. Some of these methods are directed to treating or alleviating withdrawal symptoms that persist during protracted abstinence. Examples of such symptoms include craving and disturbance in sleep or affect. Some other methods are directed to enable or help the subjects who are in protracted abstinence to reduce or refrain from cannabis use.

Thus, methods of the invention are useful to help a subject reduce or completely cease cannabis use, and alleviate symptoms of acute withdrawal syndrome. The treatment can also be directed to subjects who have already undergone acute withdrawal (cessation or substantial reduction of cannabis use) to prevent relapse or to alleviate symptoms associated with protracted abstinence. Detailed procedures for treating subjects suffering from cannabis dependence with gabapentin are described above and further exemplified in the Examples below.

### VII. Pharmaceutical Compositions and Administration

Gabapentin and the other gabapentin compounds described above may be administered directly to subjects in need of treatment. However, the gabapentin compound is preferably administered to the subjects in pharmaceutical compositions which comprise the gabapentin compound and/or other active agents along with a pharmaceutically acceptable carrier, diluent or excipient in unit dosage form. Some examples of dosage unit forms are tablets, capsules, pills, powders, aqueous and nonaqueous oral solutions and suspensions, and patches.

Pharmaceutically acceptable carriers are agents which are not biologically or otherwise undesirable, i.e., the agents may be administered to a subject along with a gabapentin compound without causing any undesirable biological effects or interacting in a deleterious manner with any of the components of the pharmaceutical composition in which it is contained. The compositions can additionally contain other therapeutic agents that are suitable for treating alcohol or cannabis dependence as described above. Pharmaceutical carriers enhance or stabilize the composition, or facilitate preparation of the composition. Pharmaceutically acceptable carriers include solvents, dispersion media, coatings, antibacterial and antifungal agents, isotonic and absorption delaying agents, and the like that are physiologically compatible. The pharmaceutically acceptable carrier should be suitable for various routes of administration described herein.

A pharmaceutical composition containing a gabapentin compound and other therapeutic agents described herein can be administered by a variety of methods known in the art. The routes and/or modes of administration vary depending upon the desired results. Depending on the route of administration, the active therapeutic agent may be coated in a material to protect the compound from the action of acids and other natural conditions that may inactivate the agent. Conventional pharmaceutical practice may be employed to provide suitable formulations or compositions to administer such compositions to subjects. Any appropriate route of administration may be employed, for example, but not limited to, oral administration, intravenous, parenteral, transcutaneous, subcutaneous, and intramuscular administration.

The gabapentin compound for use in the methods of the invention should be administered to a subject in an amount that is sufficient to achieve the desired therapeutic effect in a subject in need thereof. Typically, a therapeutically effective dose or efficacious dose of the gabapentin compound is employed in the pharmaceutical compositions of the invention. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular compositions of the present invention employed, or the ester, salt or amide thereof, the route of administration, the time of administration, the rate of excretion of the particular compound being employed. It also depends on the duration of the treatment, other drugs, compounds and/or materials used in combination with the particular compositions employed, the age, gender, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. Exemplary dosages for treating alcohol dependence or cannabis dependence in the practice of the methods of the present invention are described herein.

Pharmaceutical compositions to be employed in the methods of the present invention can be obtained commercially or prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. In some preferred embodiments of the invention, the pharmaceutical composition to be employed is a capsule for oral administration, as described in the Examples below. Such a composition comprises a therapeutically effective amount of a gabapentin or an analog compound, e.g., 300 mg, 600 mg, 900 mg, 1200 mg, or 1800 mg gabapentin, as exemplified in the Examples. Methods of administering to treat subjects suffering from alcohol dependence or cannabis dependence are described herein and specifically exemplified in the Examples below.

### EXAMPLES

The following examples are provided to further illustrate the invention but not to limit its scope. Other variants of the invention will be readily apparent to one of ordinary skill in the art and are encompassed by the appended claims.

### Example 1

### Gabapentin for Reducing Craving in Subjects with Alcohol Cue Exposure

This Example describes the results of a study to examine the effectiveness of gabapentin 1200 mg for attenuating symptoms of protracted abstinence in a non treatment-seeking sample of cue-reactive, alcohol-dependent individuals. Subjects were 33 paid volunteers with current DSM-IV alcohol dependence and a strength of craving rating 1σ or greater for alcohol than water cues. Subjects were randomly assigned to gabapentin or placebo for 1-week and then participated in a within-subjects trial where each was exposed to standardized sets of pleasant, neutral, and unpleasant visual stimuli followed by alcohol or water cues. A key aspect of the study design is exposure to alcohol cues (sight and smell of the subject's favorite alcoholic beverage) without consumption. Alcohol consumption would be expected to mask the effects of any medication that targets symptoms of protracted abstinence, as either or both could attenuate such symptoms; the effects of alcohol and the drug to be tested would therefore be inextricably confounded. The design is therefore relatively novel in combining elicitation of craving while inducing both positive and negative affect (along with interaction effects) with non-consumptive alcohol cue exposure. As such it may be particularly well-suited for testing the effectiveness of pharmacological treatments intended to normalize the neurobiological imbalances associated with protracted abstinence in alcoholics.

### A. Subject Enrolled in the Study

Subject enrollment: The study was conducted at the University of Miami School of Medicine, Miami, Fla. and approved by that institution's Institutional Review Board as conforming to the ethical standards of the 1964 Declaration of Helsinki. Subjects provided written informed consent prior to participation. Subjects were non treatment-seeking paid volunteers recruited primarily through advertisements, meeting the Diagnostic and Statistical Manual of Mental Disorders, 4th Edition (DSM-IV; American Psychiatric Association, 1994) criteria for current alcohol dependence, abstinent from alcohol on the day of the study, as verified by Breath Alcohol Concentration (BAC), not in acute withdrawal as verified by a Clinical Institute Withdrawal Assessments for Alcohol (CIWA-Ar; Sullivan et al., Br J Addict 84:1353-1357, 1989) score of <8, and meeting the definition of “cue reactive”. An individual was considered cue reactive if his or her “strength of craving” score under a neutral affect condition (see Study Overview below) was ≧1 standard deviation (3 VAS rating scale points; see measures discussion) greater for alcohol than for water cues during a mini cue reactivity session. This is similar to the criterion described by Rubonis et al. (J. Stud. Alcohol. 55:487-494, 1994) to classify cue reactors. Exclusionary criteria consisted of clinically significant medical or psychiatric disorders including depression, anxiety, or dependence on substances other than alcohol and nicotine.

Study Overview and Experimental Design: Subjects meeting admission criteria were randomized to receive gabapentin 1200 mg or placebo. Because image-based affective cue reactivity was not as strong as we had anticipated, to improve statistical power we augmented the control group with untreated individuals from three otherwise-similar lab studies. Criteria for participation were the same in all cases. Dosages (matched in the placebo condition) were titrated as follows: 300 mg morning on day 1, 300 mg morning and also evening on day 2, 300 mg morning, midday and evening on day 3, and 300 mg morning and midday followed by 600 mg evening for days 4-7. On day 7 of double-blind medication, overall measures of mood, sleep, and craving were assessed. This one-week interval of chronic dosing is intended to model and predict the critical first week on medication in a Phase II or III clinical trial, when participants' experiences with sleep and mood, or side effects, can dramatically affect treatment adherence and risk for dropout.

Subjects were given the cue reactivity protocol on day 7 of study medication. Our cue reactivity methods are described in, e.g., Mason et al., Psychopharmacol. 200:141-50, 2008. The 4-hour cue reactivity protocol included a baseline evaluation, followed by the cue reactivity procedures, and subsequent debriefing. A 3 (affective stimuli: positive, neutral, negative)×2 (beverage cue: alcohol, water) within-subjects, block-factorial design (6 repeated measures; Kirk, Experimental Design: Procedures for the Behavioral Sciences, 3rd edition. Brooks-Cole: Pacific Grove, Calif., 1995) was employed for the cue reactivity manipulation. Thus, the 6 affect-beverage trial types were: positive-alcohol, neutral-alcohol, negative-alcohol, positive-water, neutral-water, negative-water. All six mood-beverage cue combinations were presented to each subject (with order varying systematically across subjects) during the course of a single afternoon. Since order effects of cue presentation have been observed in previous studies (Monti et al., J. Abnorm. Psychol. 96:122-126, 1987; and Cooney et al., J. Abnorm. Psychol. 106:243-250, 1997), subjects were systematically assigned one of six cue order combinations, in the order they were enrolled in the study.

### B. Measures

Assessments: Standardized measures were collected prior to randomization to characterize the sample and ensure admission criteria were met. Alcohol dependence criteria were ascertained with the Structured Clinical Interview for DSM-IV (SCID; First et al., Structured Clinical Interview for DSM-IV Axis I Disorders—Patient Edition (SCID-I/P, Version 2.0), Biometrics Research Department, New York State Psychiatric Institute: New York, 1996), which was also used to exclude individuals with depressive, anxiety, or other drug use disorders. A protocol-specific standardized questionnaire was used to obtain significant drinking and substance use history, including age of first use and total years of heavy drinking (5 drinks/day for males, 4/day for females). The Timeline FollowBack Interview (TLFB; Sobell and Sobell, Timeline Follow-Back: A Technique for Assessing Self-Reported Alcohol Consumption, In: Measuring Alcohol Consumption: Psychosocial and Biochemical Methods. Litten R Z, Allen J P (eds). Humana Press: Totowa, N.J. pp 41-72, 1992) was used to determine recency, quantity and frequency of drinking in the 90 days prior to testing. The Alcohol Dependence Scale assessed dependence severity (ADS; Skinner and Horn, Alcohol Dependence Scale: User's Guide, Addiction Research Foundation: Toronto 1984). Overall subjective sleep quality and 7 components of disturbed sleep were assessed by the Pittsburgh Sleep Quality Index (PSQI; Buysse et al., Psychiatry Res. 28:193-213, 1989). The Beck Depression Inventory (BDI) assessed severity of sub-syndromal depressive symptomology (Beck et al., Arch Gen Psychiatry 4:561-571, 1961). BAC was measured by breathalyzer to confirm abstinence prior to cue exposure.

To verify a safe return to a baseline state following the cue exposure trials, the Alcohol Craving Questionnaire (ACQ; Singleton et al., Alcohol Craving Questionnaire: ACQ-Now: Background and Administration Manual. NIDA Addiction Research Center: Baltimore, Md., 1994) was administered both prior to and following the cue reactivity procedure to ensure that the trials had not resulted in increased subjective urge to drink. Similarly, the Positive and Negative Affect Scale (PANAS; Watson et al., J. Pers. Soc. Psychol. 54:1063-1070, 1988) was administered at baseline and again after the experimental session, to ensure that subjects had been adequately debriefed and had returned to their normal emotional state. The SAFTEE-G (Levine and Schooler, Psychopharmacol. Bull. 22:343-381, 1986) was obtained after the experimental session (following 1 week on drug or placebo) to assess side effects. The 49-item version of the Addiction Research Center Inventory (ARCI; Martin et al., Clin. Pharmacol. Ther. 12:245-258, 1971) was used to assess abuse potential of gabapentin in alcoholics by evaluating the similarity of its effects to known drugs of abuse.

Cue Reactivity—Subjective Measures: Alcohol craving in response to each affect-beverage condition was assessed using four individual Visual Analog Scale items (VAS; endpoints were marked with a 0 on the left indicating no craving, and a 20 on the right indicating severe craving) adapted from the ACQ (Singleton et al., 1994). In an effort to minimize response burden and attendant fatigue, a subset of the full ACQ was substituted for the entire instrument. The four items comprised the highest-loading item for each of four factors in an analysis presented by Singleton et al. (1994). These items were not specifically validated as a subscale in that study, however. The items represented expectancy for positive reinforcement (“Having a drink would make things just perfect”), strength of craving (“How strong is your craving to drink alcohol”), intent (“If I could drink alcohol now, I would drink it”), and lack of control (“It would be hard to turn down a drink right now”).

Emotional reactivity was assessed using a computerized version of the Self-Assessment Manikin (SAM; Bradley and Lang, J. Behay. Ther. Exp. Psychiatry. 25:49-59, 1994). SAM is a cartoon figure used to assess three dimensions of affect; valence (how happy or unhappy one is), arousal (excitement, possibly anxiety), and dominance (subjective sense of control). Subjects were instructed to indicate “how you are feeling right now.” Anchors for the valence dimension included “happy, satisfied, contented” versus “unhappy, sad, bored.” Arousal anchors included “stimulated, excited, jittery” versus “relaxed, calm, sluggish.” Dominance anchors included “powerful, strong, controlling” versus “powerless, submissive, controlled.” Potential responses were marked with 0 (least strong) on the left end to 20 (strongest) on the right end. Two additional VAS ratings were used to provide manipulation checks on the experimental conditions. These questions represent beverage preference (“How much did you like the beverage just given to you”) and picture emotiveness (“Watching the pictures made me feel a strong emotion”). These questions also were anchored with extreme values of 0 and 20 (20 indicating strongest emotion).

Cue Reactivity—Psychophysiological Measures: Heart rate (HR), skin conductance (SC), and facial electromyogram (EMG) were monitored throughout each experimental trial as confirmatory measures of the primary subjective measures of craving and emotion. The focus of the present analyses is on the 90-second in vivo beverage cue exposure periods. Following guidelines provided by Fridlund and Cacioppo (Psychophysiol. 23:567-589, 1986), electrodes were placed on the corrugator and zygomatic muscle regions on the left side of the face to record negative affect (frowning) and positive affect (smiling) respectively.

Affective Stimuli: Positive, neutral, and negative pictures were selected from the International Affective Picture System (IAPS; Center for the Study of Emotion and Attention, The International Affective Picture System, The Center for Research in Psychophysiology, University of Florida, Gainesville, Fla. 1994). Two sets of equivalent images were selected for each affective category (positive, negative, neutral), in order to reduce habituation across the 2 beverage conditions. Thus, 24 pictures from each affective category were used. Prior work in our lab has verified that the selected affective slides are associated with the expected affective category (Mason et al., 2008).

Procedures: The cue reactivity protocol is summarized in Table 1, and details are provided in Appendix 1. Briefly, subjects completed the baseline clinical assessments, and then were escorted to a comfortable chair in an isolated room, and sensors to record psychophysiological responses were attached. For each of the six cue combinations (alcohol/water by positive affect/negative affect/neutral affect) a computer screen displayed the appropriate emotion-evoking IAPS images, followed by placement of the subject's favorite alcoholic beverage or water on a small table close enough for the subject to see, touch, and smell. Psychophysiological, subjective craving and other ratings were obtained in the course of each such cue combination. After completing all six cue combinations, subjects were debriefed, and reassessed to ensure safe return to baseline.

Statistical Analysis: Mixed-effects modeling was used for statistical analysis. This approach produces results more general than but otherwise similar to repeated measures ANOVA (Laird and Ware, Biometrics 38:963-974, 1982; and Gueorguieva and Krystal, Arch Gen Psychiatry 61:310-317, 2004). Analyses were conducted using MLwIN software (Rasbash et al., A User's Guide to MLwiN, Institute of Education: London, 2000). Beverage (alcohol or water) and affective stimuli (positive, neutral, or negative) were treated as within-subject fixed factors, while drug condition (gabapentin or placebo) was a between-subjects fixed factor. All 33 cue reactive participants provided complete outcome data over all six cue conditions in the laboratory protocol (total observations=33×6=198). Models included all possible terms up to two-variable interactions. We decided not to include three-variable interactions because such terms complicate model interpretations without contributing to the study hypotheses. In addition we assumed that there would be no main effect of gabapentin (i.e. an effect that held on average across all cue exposure conditions), because the lab situation and outcome measures (craving, SAM, and cue checks) were designed to focus subjects' attention on differences between cue conditions and outcome changes occurring over a span of a few minutes, in contrast to more time-stable mood or motivational conditions (e.g. depression or anxiety) that might have been affected by medication. This assumption makes interpreting drug-cue interactions straightforward: coefficients of such terms tell us the extent to which medication modifies cue effects.

TABLE 1
Schedule of Procedures for Cue Reactivity Session*
Pre-Test Period

| | |
|---|---|
| 1:00 p.m. | Subject arrives; vital signs, BAC, urine toxicology screen |
| | for illicit drug use, date and time of last drink obtained, |
| | and clinical and laboratory assessments completed^{a} |
| 2:00 p.m. | Subject prepped for cue session; electrodes attached, |
| | impedance checked |
| 2:20 p.m. | Subject given instructions and cue-reactivity practice trial |

Cue Reactivity Trials

| | |
|---|---|
| 2:40 p.m. | Step 1 - Mood Induction: Subject exposed to block of 12 |
| | affective images (pleasant, unpleasant or neutral), |
| | psychophysiological recording |
| 2:45 p.m. | Step 2 - In Vivo Beverage Cue: Alcohol or water beverage |
| | placed in front of subject for 90-sec while recalling |
| | picture-induced mood, psychophysiological recording |
| 2:50 p.m. | Step 3 - Ratings: Subjects complete VAS craving, SAM, and |
| | manipulation check in presence of beverage, beverage |
| | removed from testing area after ratings completed |
| 2:55 p.m. | Repeat Steps 1 -3 for remaining affect-beverage trial |
| | combinations (6 trials total) |

Post-Test Period

| | |
|---|---|
| 3:55 p.m. | Electrodes removed, debriefing and relaxation period, |
| | post-cue session assessment of craving and affect to |
| | verify return to baseline, subject paid |
| 5:00 pm | Subject leaves |

^{a}Clinical assessments included: Clinical Institute Withdrawal Assessments for Alcohol (CIWA-Ar), Structured Clinical Interview for DSM-IV (SCID), Alcohol Dependence Scale (ADS), Alcohol Craving Questionnaire (ACQ), Positive and Negative Affect Scale (PANAS), Timeline FollowBack (TLFB)

### C. Results

Table 2 shows detailed demographic, substance use, and clinical characteristics of the sample. Most subjects were male and approximately half of the sample was non-white. Mean age was approximately 40, ranging from 19 to 59. Table 2 also provides descriptions of alcohol consumption, which was quite heavy, as intended. However, no subject reported drinking throughout the day, and most drank primarily evenings and weekends, suggesting some ability to function and carry out typical social responsibilities. Only 20% had been in previous alcohol treatment or detox. Table 2 also shows that subjects returned to baseline levels on measures of mood and craving following the cue reactivity session.

TABLE 2
Demographic and Clinical Characteristics of the Sample (n = 33)

| | | n |
|---|---|---|
| Demographics | | |
| Age, yrs | 39.7 ± 11.9 | 33 |
| Sex, male (%) | 78.8 | 33 |
| Ethnicity (%) | | 33 |
| White | 48.5 | |
| African American | 3.0 | |
| Native American | 18.2 | |
| Latino | 30.3 | |
| Drinking Characteristics | | |
| Drinks per Drinking Day^{a} | 7.2 ± 3.1 | 32 |
| Days Abstinent (%)^{a} | 20.7 ± 18.9 | 33 |
| Years of heavy drinking^{b} | 12.8 ± 10.3 | 28 |
| Most typical heavy drinking | | 28 |
| occasion (%) | | |
| Evenings or weekends | 75.0 | |
| Binges | 7.1 | |
| All day long | 0.0 | |
| Other | 17.9 | |
| Prior alcohol detox or | 21.2 | 29 |
| treatment (%) | | |
| Alcohol Dependence Scale | 16.5 ± 16.7 | 30 |
| Pre and Post Cue Testing: | | |
| Craving and Affect Measures | | |
| At Baseline Visit | | |
| Alcohol Craving (ACQ)* | | |
| Before cues | 37.2 ± 13.9 | 21 |
| After cues | 36.8 ± 15.5 | 21 |
| PANAS-Positive** | | |
| Before cues | 32.7 ± 8.2 | 27 |
| After cues | 28.9 ± 9.1 | 27 |
| PANAS-Negative* | | |
| Before cues | 13.9 ± 4.9 | 27 |
| After cues | 15.2 ± 5.8 | 27 |

*Before-after difference: N.S. (p > .05)
**Before-after difference: p < .01
^{a}During the 90 days before screening interview (Visit 1)
^{b}5+ Drinks per day for males, 4+ drinks per day for females

Safety and Tolerability: Because the control sample was augmented with cue-reactive subjects from studies in which no medication was involved, not all control subjects were assessed for adverse reactions to medication. However, of those subjects who were thus assessed, whether cue-reactive or not (n=30), we did find a significant (t(28)=2.58, p<0.02) difference in total symptom counts over the week on medication (mean 3.3, standard deviation 2.7) or placebo (mean 1.1, standard deviation 1.7). This difference was generated by symptoms of dizziness (χ²(1)=6.1, p<0.02) and fatigue (χ²(1)=6.1, p<0.02); drug and placebo groups did not differ significantly on any other symptoms. Analyses comparing ARCI scores on five drug-of-abuse groups (Morphine-Benzadrine, Pentabarbitol-Clorpromazine-Alcohol, LSD, Benzadrine, and Amphetamine) for gabapentin vs. placebo found no statistically significant differences, suggesting that gabapentin does not produce effects resembling those of drugs in these groups. Like safety and tolerability, ARCI was obtained only for a subsample of subjects (n=28, 17 treatment, 11 placebo).

Mixed Effect Models: Table 3 presents results of mixed-effect analyses for all subjective outcome measures. The models associated with each row contain three within-subject main effect terms, dummy-coded to represent beverage cue (0=water, 1=alcohol), positive affective stimuli (0=neutral, 1=positive), and negative affective stimuli (0=neutral, 1=negative), and two interaction terms, beverage cue by positive affect, and beverage cue by negative affect. Hence within-subject effects are compared to a water, neutral, or water-neutral conditions. In addition, treatment is a dummy-coded (0=placebo, 1=gabapentin) between-subjects effect. Columns in Table 3 contain parameter estimates for all effects specified in each model. Note that each model specification includes the same set of predictor variables.

Manipulation Checks: Validity of beverage and affective cues were investigated in several ways. First, we examined their effects on ratings of beverage-liking and feeling strong emotion (Table 3, rows 1 and 2). We found a statistically significant main effect of beverage cue on beverage-liking, with alcohol preferred to water; and also significant main effects of both positive and negative image exposure relative to neutral images on feeling strong emotion. Since affective stimuli preceded beverage presentation, results suggest that the affective response lasted through the period of beverage presentation. We also calculated within-subject correlations (Snijders and Bosker, Sociol. Methods Res. 22:342-363, 1994) between psychophysiological measures (heart rate, skin conductance, zygomatic, and corrugator averages calculated during in vivo beverage exposure) and all nine subjective measures including the four craving questions, three SAM questions, beverage liking, and feeling strong emotion; thus 4×9=36 such correlations were examined altogether. While these correlations were all small and most were non-significant, consistent with findings from other studies (Ooteman et al., Alcohol. Clin. Exp. Res. 30:57-69, 2006), four were statistically significant and one was trend-significant, more than would be expected by chance. Specifically, (a) heart rate was positively correlated with SAM Arousal (r=0.181, p<0.05), (b) zygomatic response was positively correlated with SAM Valence (r=0.175, p<0.05), (c) corrugator response was negatively correlated with beverage liking (r=−0.141, p<0.10), and skin conductance and heart rate were positively correlated with feeling strong emotion (r=0.212 and 0.184, respectively, p<0.05 for both). Taken together, these results suggest that the experimental conditions affected subjects as expected.

Random Effects: Variances were allowed to differ across cue combinations, and nonzero covariances between model effects were permitted, both within and between subjects. Significant effects were found for some models; however, as they are largely nuisance effects for purposes of the present study, they are not reported.

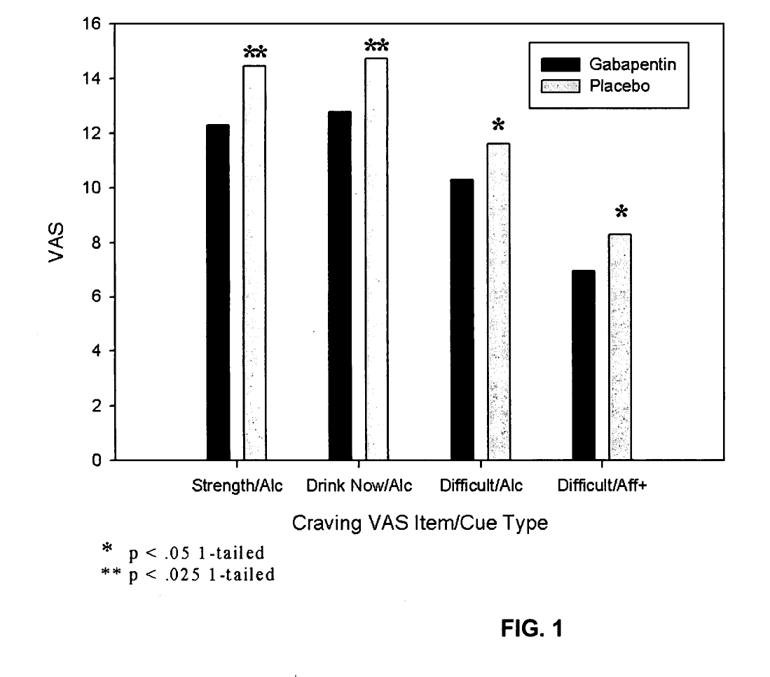

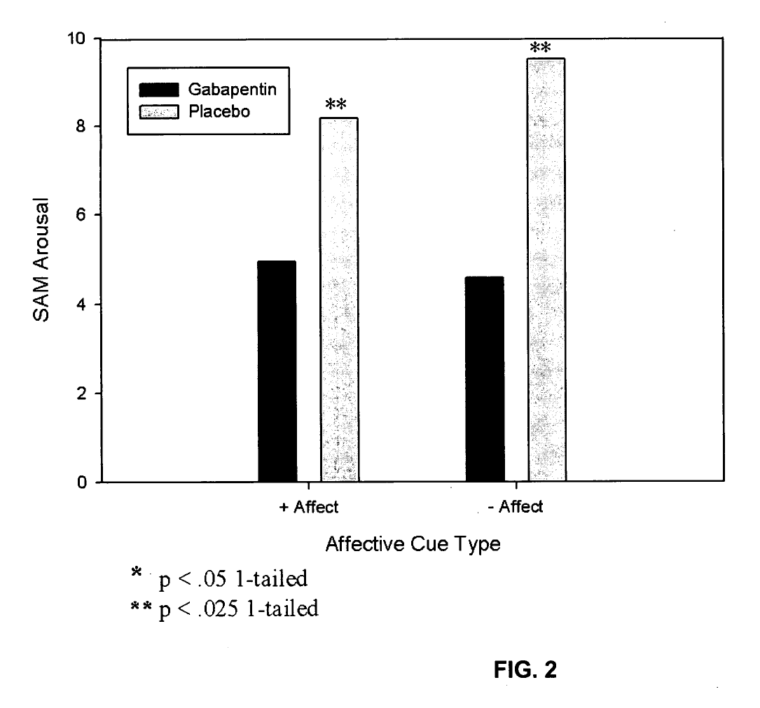

Primary study hypotheses are tested with results from the last three columns of Table 3, i.e. those involving interactions between medication and, respectively, alcohol, positive affect, and negative affect cues. These parameter estimates indicate the extent to which gabapentin alters subjects' reaction to these three types of cues, compared to placebo. Negative values imply attenuation, while positive values show augmentation. FIGS. 1 and 2 summarize the statistically significant findings concerning these effects.

We hypothesized that gabapentin would attenuate alcoholic beverage-induced craving compared to placebo. As shown in the first three sets of bars in FIG. 1 and rows 3-5 of Table 3, this hypothesis is confirmed for three of the four subjective craving measures (how strong is your urge to drink, I would drink now if I could, it would be difficult to turn down a drink now). We also hypothesized that gabapentin would attenuate affectively-induced craving significantly more than placebo. As the last set of bars in FIG. 1 shows, this hypothesis was supported for the effect of medication on positive-affect-induced craving on one craving measure (difficult to turn down a drink now). No other craving measures showed this effect for positive affect-induced craving, and none showed such an effect for negative affect-induced craving.

We further hypothesized that gabapentin would reduce arousal induced by all three types of cues. The analyses supported this hypothesis directionally for all three cue types, and the level of attenuation was statistically significant for positive and negative affective cues. FIG. 2 shows the latter two results; the full model is shown in Table 3, row 8.

Although not a major focus of the study, an additional finding is worthy of mention. Row 7 of Table 3 shows a significant and positive effect of medication on valence (larger values indicate more positive affect) induced by alcohol cue. The main effect of alcoholic beverage exposure (direction only, non-significant) was negative, indicating some negative affect or tension induced by the exposure, plausibly because subjects knew they would not be allowed to consume the alcohol. This negative affect was significantly attenuated for those subjects taking gabapentin compared to placebo, consistent with a beneficial effect of gabapentin.

Gabapentin Effects on Secondary Outcomes: Several of the Pittsburgh Sleep Quality Index component scales showed greater improvement in gabapentin than placebo groups. Subjective quality was significantly (p<0.001) better in the gabapentin group than placebo, latency (time required to fall asleep) was shorter, and efficiency (percent of time in bed the respondent actually slept) was greater (trend, p<0.06). The global PSQI sleep index was also significantly better in the gabapentin group, vs. placebo (p<0.05). Of the 7 PSQI subscales, only one did not show at least some advantage in the gabapentin group compared with placebo.

In summary, results obtained from this study provided support for the efficacy of gabapentin in attenuating craving and other symptoms of protracted abstinence known to predict relapse among alcoholics in treatment. Gabapentin significantly attenuated craving for alcohol vs. water on three of four subjective craving measures. Gabapentin also reduced positive affect cue-based craving significantly on the “difficult to turn down a drink now” rating vs. placebo. Gabapentin was found to attenuate rating of arousal for alcohol (trend), positive affect, and negative affect cues. Finally, gabapentin significantly improved several measures of sleep quality compared to placebo.

### Example 2

### Using Gabapentin to Treat Cannabis Dependence

This Example describes treatment of subjects suffering from cannabis dependence with gabapentin. Subjects with DSM-IV cannabis dependence were enrolled in a clinical study of treatment with gabapentin. Earlier, in a pilot laboratory study, the safety and efficacy of gabapentin 1200 mg/d in non treatment-seeking volunteers with current cannabis and alcohol dependence involving were examined in one week of chronic dosing with double-blind, randomly assigned active drug or placebo. Sleep disturbance and sleep quality measures showed significant improvements with gabapentin relative to placebo. There were no adverse drug complaints of more than mild severity, no drug-related study terminations, and no change from baseline in CBC, LFT, or urinalysis values. The results lend support to: 1) the safety and tolerability of our drug titration schedule and the steady state dose of gabapentin 1200 mg/d in outpatients with cannabis dependence; 2) the efficacy of gabapentin 1200 mg/d in treating disturbed sleep, a key symptom of cannabis withdrawal; and 3) our ability to recruit a sample of subjects with cannabis dependence who are willing to participate in clinical research involving medication. Based on the results from this pilot study, subjects with only cannabis dependence were then recruited and treated with gabapentin, as detailed below.

### A. Treatment Design and Subjects Recruitment

This clinical study is a 12-week randomized, double-blind, placebo-controlled, parallel group comparison of gabapentin 1200 mg/d or placebo. Subjects were 28 outpatients with current cannabis dependence who were not abstinent from cannabis more than 7 days prior to randomization. All subjects received motivational interviewing at Weeks 0-3 to facilitate a stage of readiness to change that supports setting a quit date for marijuana smoking at Week 4. Thereafter, all subjects received individual cognitive-behavioral coping skills therapy aimed at helping the patient identify triggers for cannabis use, and strategies for avoiding or coping with these triggers (Weeks 4-12). Research assessments occurred weekly through the treatment phase of the 12-week study. Post treatment follow-up assessments occurred at Week 13.

Subjects included both male and female outpatients who met DSM IV criteria for current cannabis dependence. Neither gender nor race has been found to affect the pharmacokinetics of gabapentin. The inclusion criteria for recruiting subjects are: (1) Males or females from 18-65 years of age; (2) Meets DSM IV criteria for current cannabis dependence; (3) Seeking research-based outpatient treatment for cannabis problems; (4) Smoked marijuana at least once a week in the 90 days prior to randomization; and (5) Willing to attend 12 weekly study visits and 1 follow-up visit. The criteria for exclusion are as follows: (1) Abstinent from cannabis more than 7 days at the time of randomization; (2) Active suicidal ideation; (3) Currently meets DSM IV criteria for dependence on substances, or has urine drug screen positive for substances, other than cannabis or nicotine; (4) Significant medical disorders that will increase potential risk or interfere with study

TABLE 3
Mixed-Effect Analysis Summary for Outcome Measures

| | Estimated Model Coefficients | | | | | | | | |
|---|---|---|---|---|---|---|---|---|---|
| | | | | | | | Medication Effects | | |
| | | | Induced | Induced | Bev x | Bev x | Bev x | Aff+ x | Aff− x |
| Outcome | Intercept | Beverage | Affect + | Affect − | Aff+ | Aff− | Gaba | Gaba | Gaba |

Reactivity Check

| | | | | | | | | | |
|---|---|---|---|---|---|---|---|---|---|
| 1. Like beverage | 8.27* | 7.52* | −0.38 | −1.75^{†} | 1.09 | 1.46 | −0.25 | −0.04 | 0.98 |
| 2. Feel strong emotion | 7.15* | 1.01 | 7.12* | 7.83* | −0.64 | −0.64 | 1.15 | −1.08 | −1.29 |

Craving

| | | | | | | | | | |
|---|---|---|---|---|---|---|---|---|---|
| 3. Strength | 7.33* | 7.12* | 1.46^{†} | −0.38 | −3.03* | −1.52 | −2.16^{†} | 0.25 | 0.96 |
| 4. Drink Now | 8.88* | 5.86* | 0.43 | −0.49 | −1.27 | −1.21 | −1.96^{†} | 0.16 | 0.58 |
| 5. Difficult to turn down | 7.94* | 3.69* | 0.37 | −1.55^{†} | 0.33 | 0.85 | −1.31^{††} | −1.34^{††} | 1.26 |
| 6. Make Things Perfect | 7.42* | 2.96* | 0.27 | −0.65 | −0.46 | 0.15 | 0.23 | 0.44 | 0.20 |

Subjective Emotional Reaction

| | | | | | | | | | |
|---|---|---|---|---|---|---|---|---|---|
| 7. SAM Valence | 12.79* | −1.22 | 0.11 | −2.15* | 1.94^{†} | 0.76 | 3.28* | 0.21 | 1.25 |
| 8. SAM Arousal | 6.24* | 4.16* | 1.93^{††} | 3.27* | 1.00 | −3.26 | −1.79 | −3.22^{†} | −4.92* |
| 9. SAM Dominance | 11.27* | −1.66^{†} | 0.80 | −1.50^{††} | −0.03 | 0.49 | 0.81 | 0.80 | 1.30 |

*p < .01 2-tailed
^{†}p < .05 2-tailed
^{††}p < .05 1-tailed

participation, e.g., plasma creatinine >1.4 mg/dL; (5) Sexually active female patients with childbearing potential who are pregnant, nursing or refuse to use a reliable method of birth control; (6) Meets DSM IV criteria for a major AXIS I disorder other than cannabis and nicotine dependence, including depressive and anxiety disorders, and is in need of, or currently undergoing, pharmacotherapy; (7) Inability to understand and/or comply with the provisions of the protocol and consent form; (8) Treatment with an investigational drug during the previous month; (9) Sensitivity to study drug as evidenced by adverse drug experiences with gabapentin or its ingredients; (10) Ongoing treatment with medications that may affect study outcomes, e.g., acamprosate, antidepressants, benzodiazepines, or other psychoactive medications; or (11) Subjects for whom treatment of cannabis use is being mandated by a legal authority; or (12) Subjects who plan to move out of the geographic area served by the Clinic prior to study completion.

Initial screening was conducted over the telephone (Week-2). Clinically trained study personnel used a structured interview to identify individuals likely to meet DSM IV criteria for cannabis dependence and who are seeking research-based treatment. Interested individuals who appeared eligible for the study based on this preliminary phone screen were scheduled for a face-to-face intake interview to complete the diagnostic and baseline evaluations.

During their first face-to-face interview (Week-1), potential study participants had the study explained to them and were asked to sign an IRB-approved Informed Consent, witnessed by a third party. The Structured Clinical Interview for DSM IV (SCID; First et al., 1996) were administered to determine the inclusion criteria of current cannabis dependence, and to rule out AXIS I disorders that would warrant study exclusion, e.g., depression, anxiety or psychotic disorders, or dependence on substances other than cannabis and nicotine.

The subject's demographic information, medical, and cannabis use history, and use of concomitant treatments were recorded using standardized forms. Vital signs, EKG and specimens for urinalysis, pregnancy test (if female), urine drug screen, blood chemistry, that includes gamma glutamyl transferase (GGT), and complete blood count with differential (CBC w/diff) were collected by the research nurse and prepared for same-day pick up by LabCorp for analyses. Abnormal EKG's were read and evaluated to determine if study admission is contraindicated.

The Readiness to Change Questionnaire (Heatherton et al., 1991) was used to assess change in stage of readiness to quit marijuana smoking between Weeks-1 and 4, as Motivational Interviewing was delivered at visits 0-3 to facilitate a stage of change that permits establishing a cannabis quit rate at visit 4.

Subjects who met inclusion criteria were given additional assessments (described below) that characterized the sample and measured quantity and frequency of cannabis consumption, harmful consequences of use, symptoms of cannabis withdrawal and measures used in exploratory analyses. Reasons for study exclusion were recorded for later analysis to detect any bias in the treated sample related to baseline characteristics.

### B. Assessment Measures

Study measures to characterize the sample of subjects are as follows. Fagerstrom Test for Nicotine Dependence (Heatherton et al., 1991) is a 6-item rating scale of nicotine dependence (baseline visit, self report, 2 minutes). Illicit Drug Use Index (Clayton and Voss, 1981) is a composite index of frequency and duration of illicit drug use (baseline, Study Clinician, 7 minutes). Readiness to Change Questionnaire (RTCQ) (Heatherton et al., 1991) is a 12-item questionnaire based on Prochaska and Di Clemente's stages-of-change model that is used to categorize subjects' stage of readiness to quit marijuana smoking (screening and Week 4, self report, 5 minutes). Standardized History form records demography, weight, height, medical history, and cannabis history, including age of first use, age of first regular use, years of use and number of serious prior quit attempts and treatment goals (screening visit, Research Nurse, 10 minutes). Structured Clinical Interview for the DSM IV (SCID; First et al., 1996) was used to establish the categorical diagnosis of current cannabis dependence and to rule out exclusionary Axis I disorders.

Primary Outcome Measures are as follows. Marijuana Withdrawal Discomfort Scale (WDS; Budney et al., 2001) is the sum of the 4-point severity ratings for each of the 10 most frequently reported marijuana withdrawal symptoms: anger, craving, depressed mood, decreased appetite, headaches, irritability, nervousness, restlessness, sleep difficulty, and strange dreams (every study visit, self-report, 2 minutes). Timeline Followback Interview (TLFB; Sobell and Sobell, 1992; Fals-Stewart et al., 2000) assesses the pattern and frequency of cannabis smoking at baseline and throughout the study. Due to variance in THC content and smoking dynamics, at the end of the TLFB, as per the Marijuana Treatment Project methods (Stephens et al., 2002), single-item questions assess the typical quantity of marijuana smoked per week (ounces), the typical number of joints smoked per day of use, the number of hours high per day, and the number of times a subject smoked per day (Research Nurse, 20 minutes at screening, 5 minutes at weekly study visits). TLFB data are supported by weekly urine THC/creatinine ratios, and monthly collateral informant reports. If unresolved inconsistencies between sources occur, or if the THC sample is hydrated or otherwise tampered with, then the most negative outcome will be assumed accurate. Urine THC/Creatinine ratio (Huestis and Cone, 1998) is a highly sensitive and specific quantitative analytic procedure to determine new marijuana use or abstinence. Gas chromatography-mass spectrometric levels of 11-non-9-carboxy-9-THC, the primary marijuana metabolite, are normalized to the urine creatinine concentration to reduce the variability of drug measurement attributable to urine dilution.

Secondary outcome measures are as follows. Sleep was evaluated with Pittsburgh Sleep Quality Index (PSQI; Buysse et al., 1989) which assesses subjective sleep quality and disturbance (every study visit, self report, 5 minutes). Consequences were examined with The Marijuana Consequences Checklist (adapted by Budney et al. (1998) from Stephens et al. (1993) which assesses the incidence of 26 frequently reported consequences of marijuana dependence, e.g., interpersonal problems, memory problems, and financial difficulties (Weeks 0 and 12, self-administered, 5 minutes). Craving was examined with The Marijuana Craving Questionnaire (Budney et al., 2001) which is a 10-item questionnaire that yields a total craving score and two subscale scores. Subscale 1 reflects intention and desire to smoke marijuana and anticipated pleasure; subscale 2 reflects anticipation of relief from negative affect and withdrawal (baseline and every study visit, self-report, 2 minutes). Depressive severity was analyzed with Beck Depression Inventory (BDI-II; Beck et al., 1996) which is a self-rating of severity of depressive symptoms (baseline and every study visit, 5 minutes). Safety and tolerability of study drug is examined with Systematic Assessment for Treatment Emergent Events—General Inquiry (SAFTEE-GI; Levine and Schooler, 1986) which records each adverse event and onset, duration, severity, relation to study medication and clinical action (every study visit, Research Nurse, 5 minutes).
Pharmacotherapy conditions of the treatment are as follows. Subjects were randomly assigned to receive a double-blind standardized titration schedule to arrive at a fixed dose of 1200 mg/d of gabapentin or identical placebo. All subjects received a one-week supply of medication in a blistercard package containing two placebo capsules to be taken three times a day, with 300 mg capsules of gabapentin replacing placebo capsules in the active medication group according to the following dosing schedule: Day 1—one 300 mg capsule in the evening; Day 2—one 300 mg capsule in the morning and evening; Day 3—one 300 mg capsule in the morning, at midday and in the evening. Day 4—two 300 mg capsules in the evening and one 300 mg capsule in the morning and at midday. The blistercard packaging organized the medication such that each dosing event was identified for the patient by day and time of day, with all patients taking two identical capsules three times a day, regardless of medication condition throughout the 12-week study. Subjects were titrated off active medication during Week 12 by substituting one placebo capsule for one capsule of active medication per day, in the reverse order of the initial dose titration, with all subjects entirely on placebo by the end of Week 12. Patients were instructed to return their blistercards at every study visit for drug accountability, and for review by study counselors. In the event that a subject was unable to tolerate the fixed dose of 1200 mg/d, dose reduction to the minimal therapeutic dose as per the package insert of 900 mg/d was permitted, and the excluded tablets were removed from the blistercard by the study pharmacist. Patients unable to tolerate 900 mg/d were removed from study and noted as such on the CRF reasons for termination page. Protocol-specific compliance enhancing procedures were implemented by study counselors in the case of consistently missed doses, e.g., linking missed dose to a routine activity such as mealtime or brushing teeth.

### C. Behavioral Therapy and Other Concomitant Treatment

Subjects also received behavioral therapy during the treatment period. Per The Marijuana Treatment Project (Stephens et al., 2002) and Project MATCH, Visits 0-3 will include motivational interviewing to facilitate arrival at a stage of change that supports setting a quit date for marijuana smoking at Visit 4. The motivational interview advises cessation and personalizes the risks of smoking and the benefits of stopping, assesses motivation and past experiences with quit attempts, discusses problems and barriers to stopping, assesses dependence, reassess desire and plan for change, and includes a plan for change including related goals such as relaxation approaches or an exercise program.

Visits 4-12 include weekly individual cognitive-behavioral coping skills therapy aimed at helping the patient to identify triggers for cannabis use, and strategies for avoiding or coping with these triggers. Importantly, both the motivation enhancement, and coping skills therapy have been associated with augmenting response to pharmacotherapy, but not with augmenting placebo response such that medication effects are obscured (e.g., O'Malley et al., 1992; Mason et al., 1994).

Other Concomitant Treatment: Patients were instructed to not use any medication without consulting the study physician, who will consults with the P.I. to determine the continued eligibility of the patient for participation in the study. The List of Allowed and Disallowed Concomitant Medications developed in collaboration with the P.I. for Project COMBINE were used as a reference. Disallowed medications are curtailed because they may confound the effects of the treatment conditions under study. All approved medications, e.g., NSAIDS, antihypertensives, were recorded in the case report form with any changes recorded throughout the study. Patients could attend whatever self-help groups they find beneficial. Attendance at 12-step meetings and other self-help groups were documented at every rating period.

Post Treatment Follow Up: Subjects were evaluated on study outcome measures 1 week (Week 13) following study completion to determine persistence of any treatment effects or adverse drug experiences. Procedures to maximize the likelihood of participation in the Week 13 follow-up interview included: identifying at least one (preferably more than one) “locator” person(s) to assist in tracking patients for follow-up assessments; developing a rapport with both the patient and their collateral informant, and informing them of the importance of the follow-up evaluations at the time of the intake evaluation; describing and reinforcing the patient's responsibility as a research subject; obtaining multiple back-up addresses and phone numbers, including beeper, home, cellular and work, and a preferable time of day to call, to assist in locating subjects; and arranging phone interviews if a patient refuses to come to the investigator's office. Patients who are found to have relapsed, or who display other clinically significant symptoms, will be offered referral for appropriate treatment.

Data Management: Data forms and behavioral ratings for each patient's baseline, double-blind, and follow-up visits were formatted into a case report form (CRF). Each CRF was checked for accuracy and completeness. Data from CRF's were double entered onto a computerized database format. All data were routinely printed and double checked after entry, and distributional statistics were calculated and examined to detect outlying and/or potentially inaccurate data values. All data are backed up on a weekly basis. CRF's that have been computer-entered are kept in a locked file. Data is stored and analyzed using computer facilities at the Division of Clinical Psychopharmacology (see Resources).

Sample Size: Sample size calculations were based on power curve estimates generated for a two sample t-test (α=0.05, two-tailed) on a continuous measure, i.e. the Marijuana Withdrawal Checklist. A sample size of 25 subjects per treatment group was estimated to have at least 80% power with an effect size of 0.80 to detect treatment group differences in withdrawal severity. A sample size of 25 subjects per treatment condition (total n=50) is also consistent with the small scale clinical trials of NIDA's Clinical Research Efficacy Screening Trial (CREST) program for cocaine abuse. CREST has the goal of screening medications to identify those with safety and potential efficacy that warrant further evaluation for cocaine abuse. Thus, the CREST small scale clinical trial format may serve as a reference for our developmental early Phase II clinical trial in cannabis dependence.

Data Analysis: For safety and adverse experiences, rates of treatment-emergent signs and symptoms were computed for each treatment group by body system (e.g., liver, hematologic). Changes and trends in laboratory results were examined based on shift tables and scatter plots. Relationship to exposure (dose and time) to study drugs was examined. For study discontinuation and medication compliance, Chi square analyses were used to compare the treatment groups on rate of study discontinuation due to adverse events and overall rate of study discontinuation. Rate of medication compliance were tested using a group t-test, or a non-parametric equivalent. For hypothesis testing, efficacy endpoints are derived from the Marijuana Withdrawal Checklist and the TLFB daily record of marijuana use with weekly urine toxicology verification and were chosen to facilitate comparison with published marijuana treatment studies in which comparable measures were the primary outcome variables (e.g., Stephens et al., Addiction 97:109-24, 2002).

Exploratory Analyses were performed to examine treatment group differences in: (1) specific measures of sleep (Pittsburgh Sleep Quality Index total and subscale scores), mood (BDI), and craving (Marijuana Craving Questionnaire); (2) the weekly point prevalence of marijuana use in order to determine the relative time course of anti-relapse effects, e.g. evidence of tolerance or “poop out” effects over the course of the study and follow up; (3) the consequences of marijuana use. Additional supportive analyses will examine whether: (1) plasma concentrations of gabapentin relate to study outcomes and adverse events; (2) negative affective states identified by the non-verbal computerized images of the Multidimensional Mood State Measure predict treatment outcome, risk of relapse, and periods of stable recovery (i.e., complete abstinence); (3) Assessment of sample characteristics on baseline measures identify potential predictors of responders to medication.

D. Analysis of Results Obtained from the Study

The results indicate that gabapentin treatment reduced cannabis use in the treated subjects. Specifically, cannabis use as defined by the ratio of THC to creatinine in urine showed a strong (p<0.01) main effect of drug in the expected beneficial direction. This is the most powerful indicator of reduced cannabis intake among subjects treated with gabapentin relative to placebo. Gabapentin typically had the most beneficial effects in those with the most pre-treatment consumption.

In addition, symptoms associated with drawal were also improved in subjects administered with gabapentin. For sleep related symptoms, gabapentin had a robust (p<0.01) effect on reducing daytime sleepiness and dysfunction relative to placebo. There was a trend towards a main effect of gabapentin improving sleep disturbance and sleep latency, suggesting an overall beneficial effect of gabapentin on cannabis-related sleep disturbance. This is particularly important in that our work has shown sleep disturbance to be the most enduring symptom of cannabis withdrawal, far exceeding the standard 23 day cannabis half-life period. In addition, gabapentin administration also improved mood in the treatment subjects. It was found that there was a significant interaction with pre-treatment depressive severity such that those with the highest depressive severity had the greatest improvement in symptoms from gabapentin versus placebo.

### Example 3

### Further Studies of Gabapentin in Treating Cannabis Withdrawal and Use

This Example describes results obtained from additional subjects randomized to the clinical trial described in Example 2 (resulting in a total number of 50 subjects) that was undertaken to examine effects of gabapentin on cannabis dependence. As noted above, this study involved a 12-week clinical trial of randomly assigned, double-blind treatment with gabapentin 1200 mg/d or placebo in 50 treatment-seeking non paid outpatients with cannabis dependence. It was designed to gain a preliminary estimate of the efficacy of gabapentin for reducing severity of marijuana use and withdrawal symptoms in patients with cannabis dependence.

Resolution of certain time-limited marijuana withdrawal symptoms may not be as rapid in an outpatient population with continued access to marijuana as in laboratory studies where marijuana abstinence is assured. Nevertheless acute marijuana withdrawal symptoms in outpatients would be expected to have a time-limited course that is related to diminished use. Such symptoms may be found on the Marijuana Withdrawal Checklist (MWC; Budney et al., 1999) and on certain components of the Pittsburgh Sleep Quality Index (PSQI; Buysse et al., 1989). Other symptoms, such as disturbances in mood, sleep quality and marijuana craving, have been observed beyond acute withdrawal and may reflect more protracted dysregulation in brain emotional systems that could motivate resumed marijuana use. Based on its neuromodulating mechanisms of action, gabapentin is further hypothesized to have a beneficial effect on such protracted symptoms of marijuana abstinence with a related effect on marijuana use. The overall hypothesis under test was that gabapentin would improve symptoms of cannabis withdrawal, such as disturbances in affect and sleep, and as a result, decrease severity of cannabis use.

Methods: A 12-week, double-blind, placebo-controlled study was conducted with random assignment to gabapentin 1200 mg/d or placebo in 50 outpatients with current cannabis dependence. Concomitantly, motivational interviewing was provided at Visits 0-3 to facilitate setting a quit date at Week 4, and manual-guided relapse prevention cognitive behavioral therapy was given weekly thereafter, at Visits 4-12. Efficacy endpoints for marijuana use were derived from the daily record of cannabis use obtained by the Timeline Followback Interview (TLFB; Sobell and Sobell, 1992; Fals-Stewart et al., 2000) with weekly urine toxicology confirmation (analyzed as the THC to creatinine ratio [THC/Cr]). Withdrawal symptoms were assessed with the MWC, PSQI, and Beck Depression Inventory (BDI-II; Beck et al., 1996).

Statistical Plan: Mixed-effect modeling (MEM; Laird and Ware, 1982; Snijders and Bosker, 1999) with iterative GLS estimation using MLwIN 2.1 software (Rasbash et al., 2000) was applied to tests of primary hypotheses. All models controlled for baseline values of the variable of interest, thus making the model essentially a model of change in a given variable over time. Longitudinal within-subjects designs included two different types of treatment effects. Treatment effects that do not involve time were termed intercept effects, because for a given subject they add (or subtract) the same value to the outcome variable at all time points over the course of treatment. Slope effects were also used and are important in understanding treatment effects because they indicate the extent to which treatment affects the rate of change in the outcome over time, i.e. the 12 weeks of treatment. Note that mixed-effect modeling can in some respects be viewed as a generalization of ANOVA approaches to analysis. This is explained at greater length in the section below on proposed analyses; however, MEM uses a linear regression (alternatively, “effects”) representation of the analysis model (e.g. Kirk, 1995). Hence, as with most regression models, single-parameter statistical tests of effects take the form of t-statistics.

Subject recruitment: Subjects were recruited by face-to-face intake evaluation of 83 individuals to yield the desired sample size of 50 randomized subjects. Primary reasons for non randomization of screened subjects were the presence of excluded Axis I disorders (n=12), medical disorders (n=4), or failure to return to clinic (n=9):

Baseline Characteristics: Treatment groups did not differ on any baseline demographic or clinical variable. Randomized subjects included Caucasian (76%) and minority (24%), males (88%) and females (12%) with a mean age of 33.9 (±9.7) years. All but 6% had 12+ years of education. Subjects typically began smoking marijuana at 14.5±3.5 years of age, had an average of 11.6±8.0 years of daily marijuana smoking, and were smoking an average of 11.0±18.4 g/wk of marijuana with a mean THC/Cr of 673.1±606.3 at the time of randomization. On average, subjects met criteria for 6±1.0 of 7 possible DSM IV criteria (of which 3 are required to meet diagnostic criteria) for current cannabis dependence. Subjects reported little or no use of alcohol or other drugs over the past year and only 22.4% were currently cigarette smokers. Family history was positive for alcohol or drugs in 43.8% of the sample.

The majority of subjects had no prior treatment attempts ( x̅ number of prior treatments=0.22±0.4), and were abstinent 0.86±1.6 days in the week prior to randomization. Subjects had a MWC mean score of 14.2±8.1 at time of randomization. Subjects endorsed an average number of 9.7±4.6 withdrawal symptoms at a 1.5±0.3 mean level of severity on the MWC, with marijuana craving (97.9%), nervousness (75.0%), decreased concentration (60.4%), irritation (60.4%), fatigue (58.3%), difficulty sleeping (56.3%), depression (52.1%), restlessness (52.1%) and yawning (52.1%) reported by more than half the sample. Unlike one prior report, marijuana withdrawal severity did not vary on the basis of family history of drugs and alcohol problems (Agrawal et al., 2008) or on the basis of other baseline variables, e.g., males vs. females.

Compliance and Treatment Retention: Mean rate of medication compliance, defined as number of pills taken divided by number prescribed, was 93.5% and was identical across treatment groups. Rate of study completion (36%) and average time on study (6.4 weeks) likewise did not differ between treatment groups. Twelve subjects were lost to follow-up immediately after randomization (Week 0) and did not return the following week. Excluding such rapid drop-outs (dropped before Week 1) from this analysis, then mean time on study was 8.40 weeks. Reasons for early termination in subjects returning for ≧1 follow up visit did not differ between groups and included: moving or schedule conflicts related to returning to work or school (n=3), no longer wanted medication (n=3), repeated rescheduling with ≧2 weeks without medication (n=2), non study-related medical events (infection, accident) requiring disallowed medication, e.g., Vicodin (n=2).

Safety and Tolerability of Study Medication. There were no deaths or terminations due to drug-related adverse events (AE's). Placebo treated subjects complained of 8.9±11.6 AE's vs. gabapentin 4.0±6.7 (p=0.11), and had significantly (p=0.04) more complaints of dizziness (n=22) than gabapentin subjects (n=0) (Table 4). Placebo subjects also had ≧2 times more complaints of fatigue, nausea, decreased appetite, and “other” AE's than gabapentin subjects, although these differences were not statistically significant. The average severity of AE's for both treatment groups was mild (2.0±0.7, 2=mild).

TABLE 4
Adverse events reported by ≧10% of subjects

| | Gabapentin | Placebo | |
|---|---|---|---|
| Adverse Events (AE's) | % | % | p |
| Depression | 30 | 28 | 1.00 |
| Fatigue | 15 | 39 | .14 |
| Nervousness | 25 | 28 | 1.00 |
| Headache | 20 | 33 | .47 |
| Nausea | 15 | 33 | .26 |
| Increased Appetite | 15 | 28 | .44 |
| Decreased Appetite | 10 | 28 | .22 |
| Abdominal Pain | 25 | 11 | .41 |
| Other | 5 | 22 | .17 |
| Dizziness | 0 | 22 | .04* |

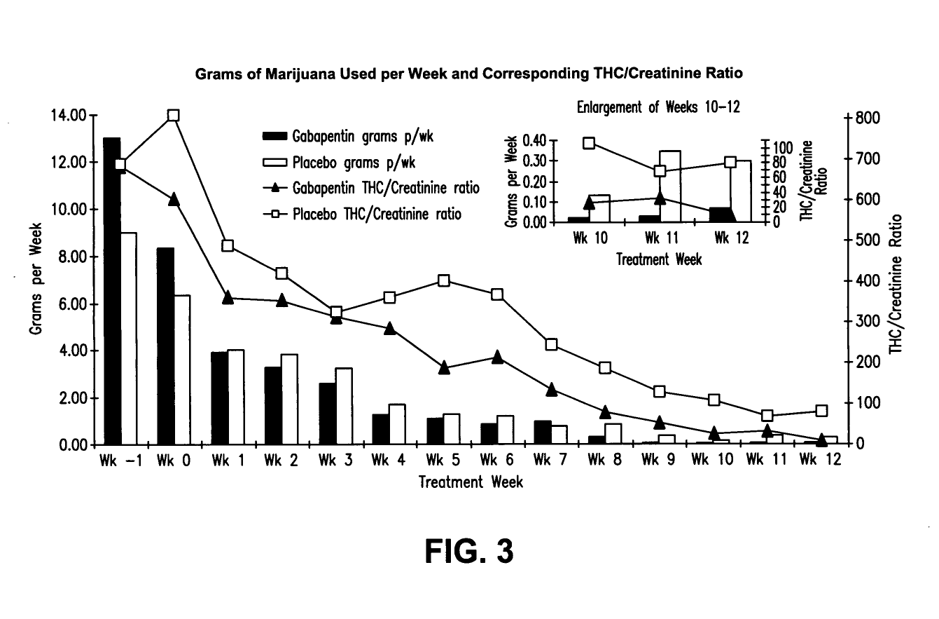

Consumption: Two primary measures were used to measure consumption: THC/Cr obtained from weekly urine assays, and grams used per week (g/wk), obtained from the TLFB interview. Overall sample means by week prior to and during treatment are presented in FIG. 3. Note that subjects typically began decreasing their use at the time of their initial call to the clinic, which corresponds to the two weeks prior to randomization (Weeks-1 and 0).

The general pattern of drug-placebo differences is readily seen in FIG. 3 to support the hypothesis that gabapentin would be associated with greater reduction in marijuana use than placebo. A mixed effects model for THC/Cr found a significant interaction between time and treatment, showing that rate of decrease in use was greater for gabapentin compared to placebo. Additionally a 3-way interaction was observed: individuals in the gabapentin condition decreased their use at a faster rate than those in the placebo group if their pre-baseline use was relatively high (t=2.51, p<0.02). Finally, there was a trend-significant (t=1.42, p<0.08 1-tailed) effect of treatment on intercept suggesting lower use in the gabapentin group vs. placebo post randomization over all 12 weeks of treatment, on average. Thus, gabapentin was found to affect both average use (intercept) and rate of decrease in use (slope) as hypothesized. A similar pattern was obtained in the mixed-effects model for g/wk. For this outcome, there were no significant slope effects, but the attenuating effect of treatment on intercept was clearly present (t=−2.68, p<0.01). Thus, the results suggest that these two quite differently-obtained consumption measures are consistent with hypothesized effects of treatment, and with each other. Additionally, change in THC/Cr was positively correlated with changes in PSQI, MWC, BDI and craving during treatment.

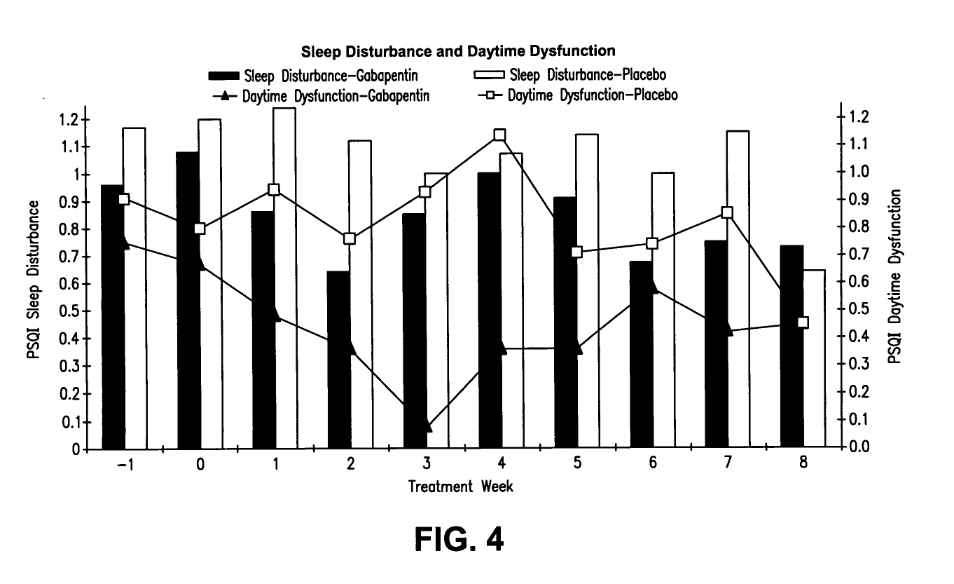

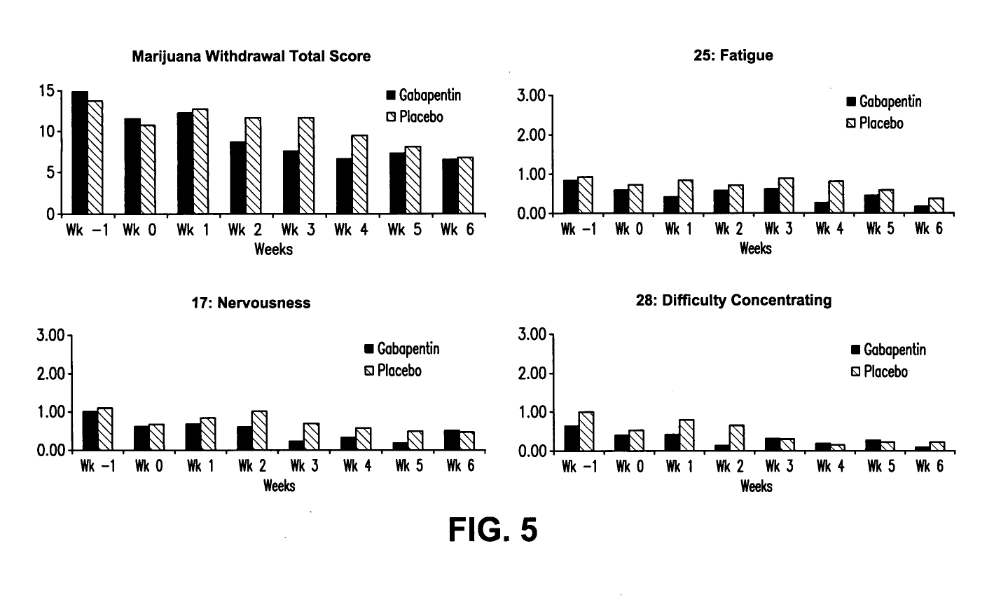

Acute Withdrawal Symptoms: Results shown in FIG. 4 suggest that there is an early improvement in two important components of sleep functioning: sleep disturbance and daytime dysfunction. The graph shows larger improvements in these indices during the first few weeks of treatment for the gabapentin group vs. placebo, an advantage which is maintained through about Week 5 or 6, when the two groups become statistically indistinguishable. The mixed-effect statistical analysis for sleep disturbance found a significant drug effect on intercept appearing in Week 1 (t=2.21, p<0.02), confirming the average lower levels of sleep disturbance for gabapentin vs. placebo. A positive slope effect for gabapentin, however, showed the two groups to come together during the later weeks of treatment. A similar pattern was found for daytime dysfunction, with the early attenuation of symptoms in the gabapentin group vs. placebo (t=2.26, p<0.025) giving way to rough equality in the later weeks. Similar early effects of gabapentin were also observed for the following outcome measures: PSQI global score and components measuring subjective sleep quality, duration, and efficiency, and the Marijuana Withdrawal Checklist total score and individual items measuring nervousness/anxiety, fatigue, and decreased concentration (see, e.g., FIG. 5).

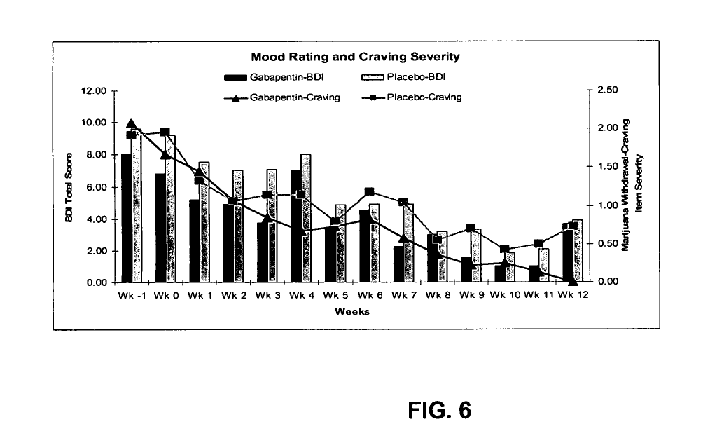

Protracted Withdrawal: A number of additional withdrawal symptoms were improved by gabapentin relative to placebo, but the effects were not apparent until late in treatment (FIG. 6). These included mood (measured by BDI), marijuana craving, and the PSQI latency subscale. In mixed effect models, gabapentin subjects' mood, craving and sleep symptoms resolved at a faster rate, especially among those with the highest baseline severity. For BDI, the three-way interaction amongst treatment, time, and baseline mood was significant (t=1.96, p<0.05), indicating an especially strong treatment effect among subjects with high baseline severity.

Similarly, subjects in the gabapentin condition reduced marijuana craving at a faster rate than placebo (t=2.0, p<0.05) and this effect was strongest for those with the highest craving at baseline (t=2.63, p<0.01). PSQI sleep latency (difficulty falling asleep) was attenuated both immediately (t=2.07, p<0.05) and also at a faster rate over time (t=2.38, p<0.01) for subjects with the highest baseline latency.

### Example 4

### Using Gabapentin to Treat Alcohol Dependence

This example describes the study of gabapentin treatment of subjects suffering from alcohol dependence. A 12-week, double-blind, placebo-controlled, dose-ranging study was conducted with random assignment to gabapentin 900 mg/day, 1800 mg/day, or placebo. Subjects are outpatients with alcohol dependence. The 3 pharmacological treatment conditions were administered in conjunction with manual-guided behavioral counseling that incorporates strategies to increase motivation, abstinence, and medication compliance. Counseling and research assessments occurred weekly throughout the 12-week treatment phase. Post-treatment assessments occurred at Weeks 13, 24, and 36. The study demonstrated safety and tolerability of gabapentin in subjects with alcohol dependence who received either escalating or steady state doses.

### A. Subjects Enrollment and Relevant Methods

Subjects are male and female outpatients over 18 years of age. There is no pharmacokinetic reason to exclude healthy older subjects with plasma creatinine ≦1.4 mg/dL. Therefore, there is no upper age limit. Similarly, neither gender nor race has been found to affect the pharmacokinetics of gabapentin and males and females and minority subjects were included. All subjects met DSM-IV criteria for current alcohol dependence.

The criteria for inclusion are as follows: (1) Males or females over 18 years of age; (2) Meets DSM-IV criteria for current alcohol dependence; (3) Seeking research-based outpatient treatment for alcohol problems; (4) Abstinent from alcohol a minimum of 3 days to a maximum of 30 days at the time of randomization; and (5) Willing to attend 12 weekly study visits and 3 follow-up visits. The criteria for exclusion are as follows: (1) Currently meets DSM-IV criteria for dependence on substances other than alcohol, nicotine or caffeine; (2) Significant medical disorders that will increase potential risk or interfere with study participation, e.g., plasma creatinine clearance >1.4 mg/dL; (3) Sexually active female subjects with childbearing potential who are pregnant, nursing or refuse to use a reliable method of birth control; (4) Meets DSM-IV criteria for a major AXIS I disorder other than alcohol dependence, including depressive and anxiety disorders, and is in need of, or currently undergoing, pharmacotherapy; (5) Inability to understand and/or comply with the provisions of the protocol and consent form; (6) Treatment with an investigational drug during the previous month; (7) Sensitivity to study drug as evidenced by adverse drug experiences with gabapentin or its ingredients; (8) Ongoing treatment with disulfuram (Antabuse), naltrexone (ReVia®), acamprosate (Campral®) or other medications that may affect study outcomes, e.g., anticonvulsants; (9) More than one month of abstinence prior to randomization; (10) Subjects who require medicated detoxification (Note: Subjects may proceed with study evaluation after completion of detoxification); (11) Subjects for whom treatment of alcoholism is being mandated by a legal authority; (12) Unable to identify at least one collateral informant to verify drinking status at baseline and monthly during study, and to assist in tracking subjects for follow-up assessments; or (13) Subjects who plan to move out of the geographic area served by the Clinic prior to study completion.

Subjects were screened with the following procedures. Initial screening was conducted over the telephone (Week-2). Clinically trained study personnel used a structured interview to identify individuals likely to meet diagnostic criteria for alcohol dependence and who are seeking research-based treatment for this disorder. Interested individuals who appear eligible for the study based on this preliminary phone screen were scheduled for a face-to-face intake interview to complete the diagnostic and baseline evaluations.

During their first face-to-face interview (Week-1), potential study subjects had the study explained to them and were asked to sign an IRB-approved Informed Consent, witnessed by a third party. The subject's demographic information, medical and alcohol use history, and use of concomitant treatments were recorded using standardized forms. At the baseline (Week 0) visit, study physicians reviewed all laboratory work, EKG's, and cardiologist reports, vital signs, medical histories, use of illicit substances and concomitant treatments, and performed a physical exam to medically clear an individual for admission to study. Subjects who meet inclusion criteria were given assessments (described below) to characterize severity of alcohol dependence, quantity and frequency of alcohol consumption, and measures used in exploratory analyses. Reasons for study exclusion were recorded for later analysis to detect any bias in the treated sample related to baseline characteristics.

The following measures were employed to assess and examine the enrolled subjects. Alcohol Dependence Scale (ADS; Skinner et al., 1984) provides a reliable and valid quantitative measure of severity of alcohol dependence (screening visit, self report, 5 minutes). Breath Alcohol Concentrations (BAC) were obtained at every study visit to confirm self report of abstinence. Subjects with positive BAC's waited until BAC is normal or are rescheduled and accompanied home by a friend or taxi driver (Research Nurse, 2 minutes). Fagerstrom Test for Nicotine Dependence (FTND; Heatherton et al., 1991) is a 6-item rating scale of nicotine dependence (screening visit, self report, 2 minutes). Illicit Drug Use Index (Clayton and Voss, 1981) is a composite index of frequency and duration of illicit drug use (screening visit, Study Clinician, 7 minutes). Structured Clinical Interview for the DSM-IV (SCID; First, 1996) was used to establish categorical diagnoses of current alcohol dependence and depressive disorder and to rule out other major AXIS I disorders (baseline, Study Psychiatrist, 45 minutes).

Primary outcome of the treatment was assesses with the following measures. Timeline Followback Interview (TLFB; Sobell and Sobell, 1992) provides quantity and frequency estimates of alcohol intake at baseline and throughout the study using a standard drinks format (Research Nurse, 20 minutes at screening and Week 24 and 36 follow-up visits, 5 minutes at weekly study visits). Note that no one method has been found to reliably and validly detect drinking that occurs between study visits. Therefore, TLFB data is supported by weekly breathalyzers and monthly collateral informant reports and GGT. If unresolved inconsistencies between sources occur, then the most negative outcome will be assumed accurate.

Secondary outcome of the treatment was assessed with the following measures. Alcohol Craving Questionnaire-Short Form (ACQ-SF; Singleton et al., 1994) provides a quick assessment of current drinking urges, difficulty resisting urges and anticipation of positive outcome or relief from negative affective state by drinking (baseline and every study visit, self report, 1-2 minutes). Beck Depression Inventory (BDI; Beck et al., 1961) is a self-rating of severity of depressive symptoms (baseline and every study visit, 5 minutes). Gamma glutamyl transferase (GGT) is used as a biochemical marker of alcohol abstinence or relapse. Pittsburgh Sleep Quality Index (PSQI; Buysse et al., 1989) assesses subjective sleep quality and disturbance (every study visit, self report, 5 minutes). Systematic Assessment for Treatment Emergent Events—General Inquiry (SAFTEE-GI; Levine and Schooler, 1986) recorded each adverse event and onset, duration, severity, relation to study medication and clinical action (every study visit, Research Nurse, 5 minutes).

Exploratory outcome of the treatment was examined with the following measures. Multidimensional Mood State Measure (MMSM; Koob et al., unpublished) are sets of computerized images used to non verbally determine negative cognitive states hypothesized to be associated with alcohol abstinence and risk for relapse. The MMSM has extensive normative data (N>600) and is currently used in our lab study of medication development for protracted abstinence and is used on an experimental basis in the present study as potential predictor of treatment response, relapse and stable recovery (baseline and every study visit, self-administered, 5 minutes).

Pharmacotherapy conditions of the treatment were as follows. Subjects were randomly assigned to receive a double-blind standardized titration schedule to arrive at a fixed dose of 900 mg/d or 1800 mg/d of gabapentin or identical placebo. All subjects received a one-week supply of medication in a blistercard package that organizes the medication such that each dosing event is identified for the subject by day and time of day, with all subjects taking two identical capsules three times a day, regardless of medication condition throughout the 12-week study. Subjects are titrated off active medication during Week 12. Subjects are instructed to return their blistercards at every study visit for drug accountability, and for review by study counselors. Protocol-specific compliance enhancing procedures were implemented by study counselors in the case of consistently missed doses, e.g., linking missed dose to a routine activity such as a mealtime or brushing teeth. Throughout the study, there were no deaths or serious drug-related adverse events.

Statistical analysis of data obtained from the study was performed as follows. All statistical tests were two-sided and had an alpha level of 0.05. The repeated measures design used in this study included assessments for baseline and a treatment phase which was 12 weeks in length. Follow-up visits occurred at weeks 13, 24 and 36. The baseline characteristics of the treatment groups were compared by analysis of variance for continuous variables and chi-square analysis for categorical variables. All baseline demographic and clinical measures were tested for correlation with outcome measures. Any baseline measure that differed between groups at baseline or that was significantly related to outcome measures was evaluated as a potential covariate.

The efficacy of gabapentin was evaluated in the intention-to-treat (ITT) population that included all randomized subjects for whom any post baseline drinking data were available. Due to the nature of the ITT population and the preliminary status of the analyses, missing data had to be considered for analysis, therefore a mixed effects model (MEM) was utilized for the drinks per drinking day, cumulative abstinence duration (CAD), and all secondary outcome measures. Linear dose effect of treatment was also evaluated. Estimates for latency to first drink were obtained using the Kaplan-Meier method, and survival curves for treatment groups were compared using the log rank test. If covariates were included, Cox Regression was performed on the latency variable.

B. Preliminary Analysis of Recruited Subjects with Alcohol Dependence

Recruitment, subject flow, and numbers analyzed: Subjects were recruited between April 2004 and August 2006. A total of 122 subjects attended a screening visit, and 73 (60%) of those subjects met study criteria and underwent randomization; 34 (28%) did not meet admission criteria and 15 (12%) were not interested. Subjects received their first dose of double-blind medication under observation in the clinic. Thus, baseline and safety data are available for all 73 randomized subjects (placebo n=25, 900 mg/d n=25, 1800 mg/d n=23). The ITT population is used for all efficacy analyses and includes all randomized subjects for whom any outcome data are available. There were no significant differences between groups on rates of study completion or length of time in the treatment phase.

Demographic and clinical characteristics: Subjects' baseline characteristics are presented in Table 5. No baseline variable differed significantly across groups. Only 21 (29%) of subjects had a treatment goal of total abstinence, and this was not different between treatment groups. A lifetime history of cannabis use was reported by 88%, cocaine use by 66%, psychedelics by 56%, stimulants by 52%, sedatives by 33%, opiates by 27% and heroin by 7%. Of those for whom data were available, within the past year, 31% reported cannabis use, 8% cocaine use, 17% sedative abuse, and other illicit drugs were used by 5% of subjects. Treatment groups did not differ based on substance use.

TABLE 5
Demographic and Baseline Characteristics*

| | | Gabapentin | Gabapentin |
|---|---|---|---|
| | Placebo | 900 mg/d | 1800 mg/d |
| Characteristic | (n = 25) | (n = 25) | (n = 25) |
| | No. (%) | | |
| Men | 12 (48) | 17 (68) | 16 (69.6) |
| White Non Hispanic | 20 (80) | 16 (64) | 18 (78.3) |
| Lives alone | 8 (32) | 4 (16) | 9 (39.1) |
| Employed full-time | 14 (56) | 15 (60) | 10 (43.5) |
| ≧2 prior treatments or | 6 (24) | 8 (32) | 2 (8.7) |
| detoxifications | | | |
| Alcohol Dependence | 4 (16) | 9 (36) | 3 (13) |
| Scale score (≧22§) | | | |
| Parental alcoholism | 8 (32) | 13 (52) | 9 (39.1) |
| | Mean (SD) | | |
| Age, year | 46.68 (10.2) | 40.8 (12.25) | 44.27 (11.72) |
| Range | 25-66 | 23-65 | 24-65 |
| Drinks per week† | 41.09 (25.71) | 44.76 (24.61) | 38.99 (20.88) |
| Drinking days per | 5.05 (1.86) | 5.34 (1.68) | 5.10 (2.0) |
| week† | | | |
| Years of heavy | 13.6 (8.6) | 13.54 (11.3) | 15.52 (11.03) |
| drinking | | | |
| Clinical Global | 3.88 (.74) | 3.96 (.62) | 3.74 (.54) |
| Impression score‡ | | | |
| γ-glutamyltransferase, | 38.92 (33.07) | 56.6 (44.44) | 45.22 (27.36) |
| (IU/L)∥ | | | |

*There were no statistically significant differences between groups.
†Mean number of standard drinks per week or drinking days per week during the 90 days prior to randomization.
‡The range for the Clinical Global Impression score is 1 to 7, with higher scores indicating greater severity of current alcohol dependence.
§The scores on the Alcohol Dependence Scale can range from 0 to 47, with scores ≧22 indicating a substantial to severe level of lifetime dependence.
∥The normal range for γ-glutamyltransferase is 0 to 65 U/L.

### C. Primary Outcome of Treating Alcohol Dependence: Reduced Drinking

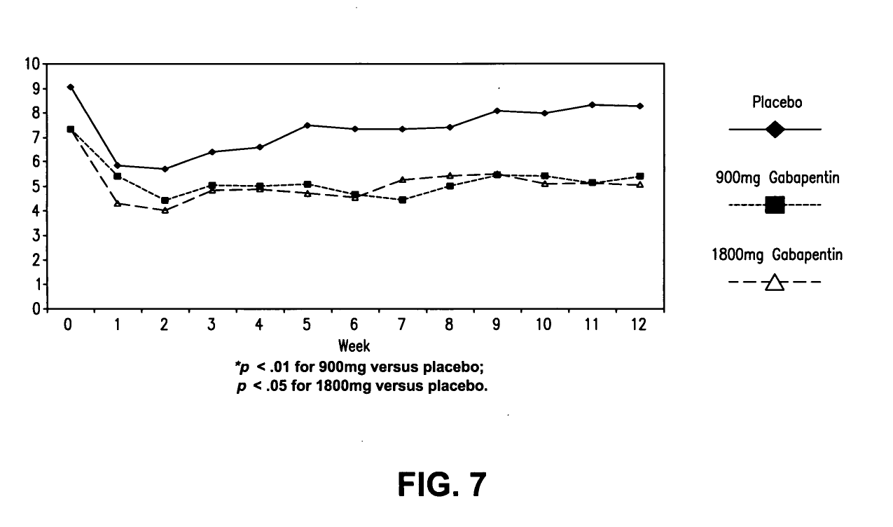

The effect of gabapentin treatment on alcohol drinking in the recruited subjects was examined. The number of drinks per drinking day (DDD) across treatments is shown in FIG. 7. Data were square-root transformed prior to MEM analysis, hence the parameter estimates and standard errors based on the transformed values are reported. The average DDD predicted during treatment for subjects on placebo whose baseline drinking was at the average for the whole sample (all three groups) was 2.517² (SE=0.120). For every drink consumed at baseline, the predicted increase in drinks consumed over the treatment period was 0.578² DDD per week for placebo (SE=0.079, p<0.01). A significant decrease in DDD per week was found for the 900 mg/d gabapentin group, with a predicted 0.385² fewer drinks per drinking day, on average, than the placebo group, SE=0.146, Cohen's⁵² d=0.385/0.47=0.82, p<0.01. Finally, the predicted value for the 1800 mg/d gabapentin group was 0.328² fewer drinks than the placebo group. This was also significant, SE=0.152, Cohen's d=0.328/0.46=0.71, p<0.05. There was no significant difference between the 900 mg/d and 1800 mg/d doses of gabapentin. There was a significant main effect of sex (even controlling for baseline DDD), such that women drank 0.352² fewer drinks (SE=0.130, p<0.01) post-baseline than men, regardless of treatment condition. Additional analyses found no evidence of interaction between sex and drug treatment—the treatment was effective for both men and women. Finally, baseline ADS, CIWA, BDI and days abstinent were not significant as covariates.

Rate of cumulative abstinence duration (CAD) was approximately normally-distributed in all three treatment groups. A one-way ANOVA did not show a significant effect of treatment, F(2, 69)=0.411, p=0.664. Levene's test showed equal variances. Correlations were performed between CAD over all weeks and baseline characteristics of interest. None were significantly correlated. Latency to first drink was analyzed using Kaplan-Meier estimates, and rates were compared using the log rank test. Results showed no significant differences in rate of survival between groups, χ²_{LR}=0.12, p=0.7341.

### D. Secondary Outcome: Alleviation of Symptoms of Protracted Alcohol Abstinence

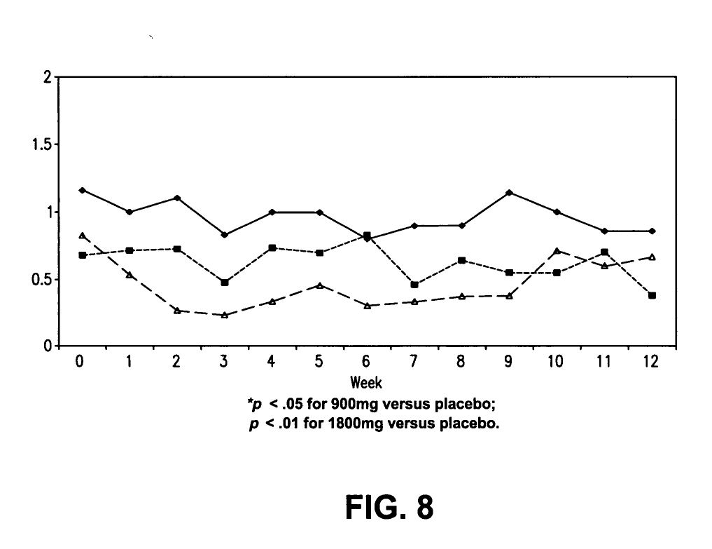

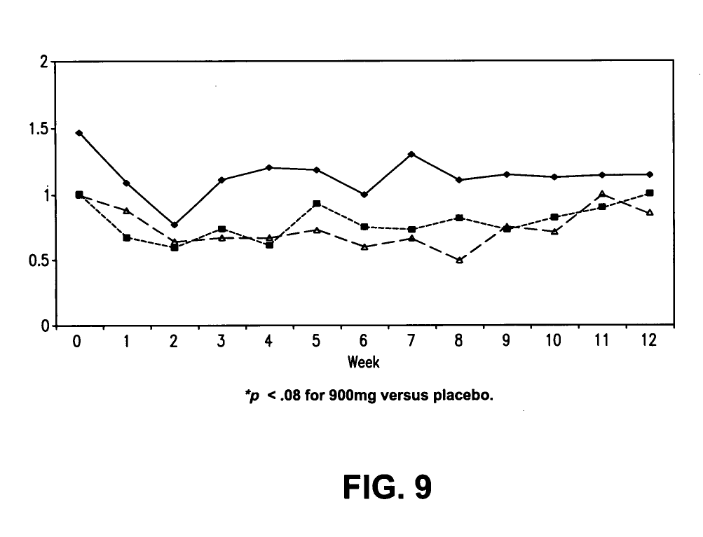

Treatment of subjects with gabapentin noted above have also alleviated symptoms associated with protracted abstinence. Significant effects showing improved sleep were found for sleep disturbance and daytime dysfunction (d=−0.50, p<0.01, and −0.27, p<0.05, respectively) for the 900 mg/d gabapentin group. Daytime dysfunction was also significantly attenuated with 1800 mg/d gabapentin, d=−0.43, p<0.01, the only linear dose effect found (FIG. 8). No significant effects of drug treatment were found for sleep efficiency or use of sleep medication, although parameter estimates were in the hypothesized (improved) direction for the 900 mg/d group. Finally, there was no significant effect of treatment on subjective sleep quality (FIG. 9) or PSQI summary score, but a trend in the hypothesized direction was observed for the 900 mg/d group, p<0.08. This is not surprising given the relatively consistent effects of 900 mg gabapentin reported above.

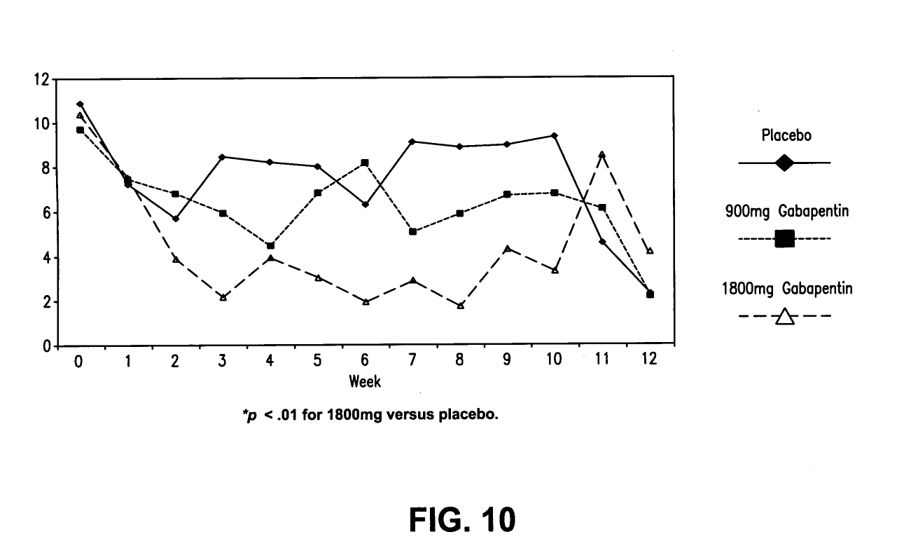

The treatment also improved mood in the subjects receiving the 1800 mg/d dosage. It was found that the treatment significantly attenuated negative affective symptoms relative to placebo, as measured by the BDI, d=0.33, p<0.05 (FIG. 10). There was no significant effect of the 900 mg/d dose on BDI total score nor an effect of covariates, e.g., sex, age, ADS, CIWA or prior detoxes/treatments ≧2 on these results.

The finding of a significant effect of treatment on number of drinks consumed per day for each gabapentin dose condition relative to placebo, but not on latency to first drink or on number of abstinent days per week, may be a function of the small proportion of subjects having a treatment goal of abstinence (29%). Alternatively, the interim analyses may be under powered to detect a drug effect on these additional outcomes, or gabapentin may have a specific effect on drinking quantity and not on abstinence, similar to naltrexone.

Importantly, unlike prior medications studied to date for alcohol dependence, gabapentin reduced both drinking quantity and key symptoms of protracted abstinence that are common precipitants of relapse, i.e., disturbances in mood and sleep. Thus, gabapentin may have a unique dual role in supporting recovery in alcohol dependence that reflects its “normalizing” mechanism of action in brain systems commonly dysregulated in early recovery, i.e., GABA and glutamate.

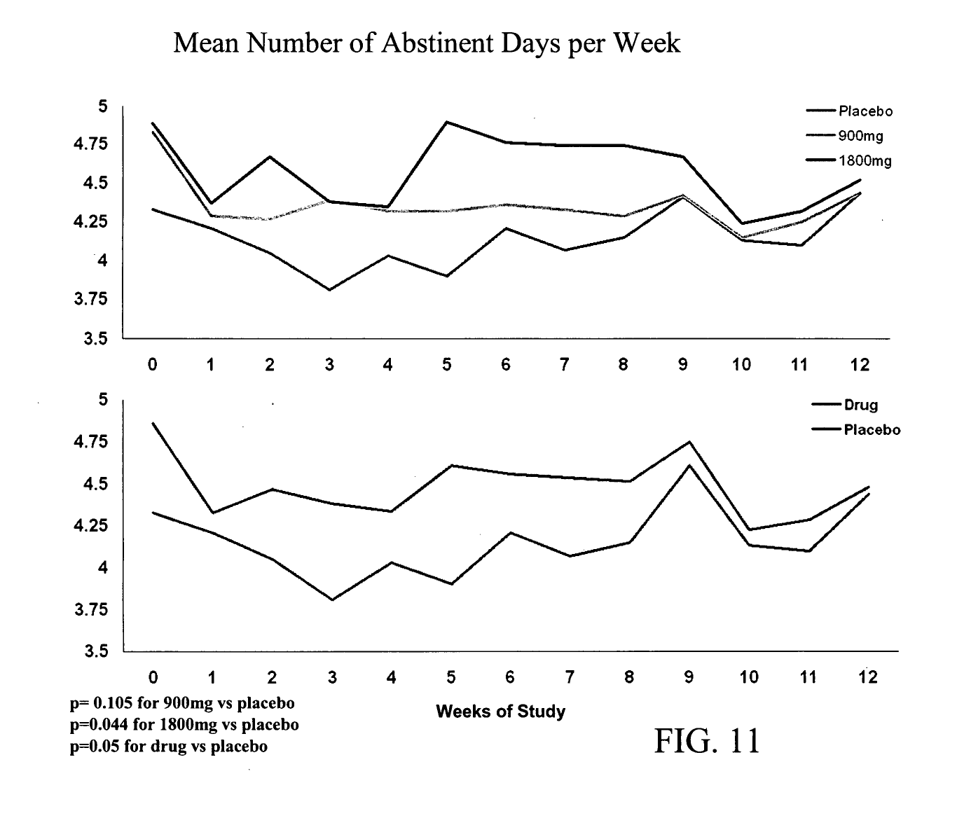

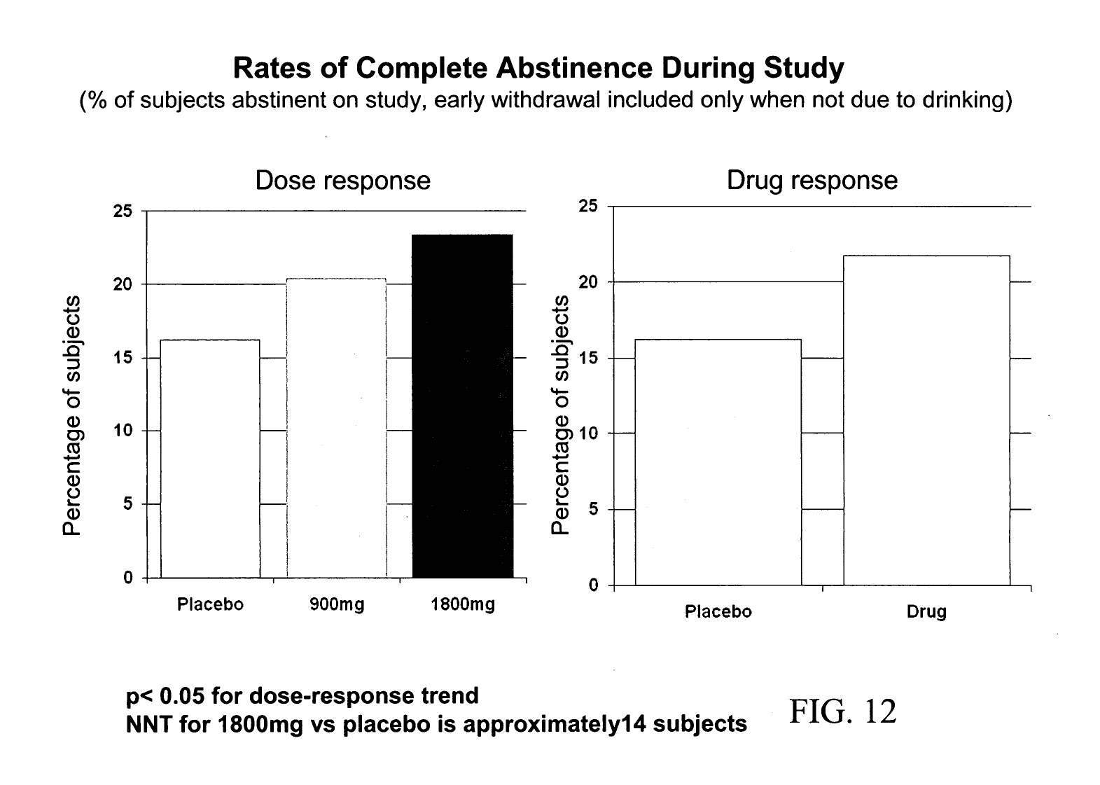

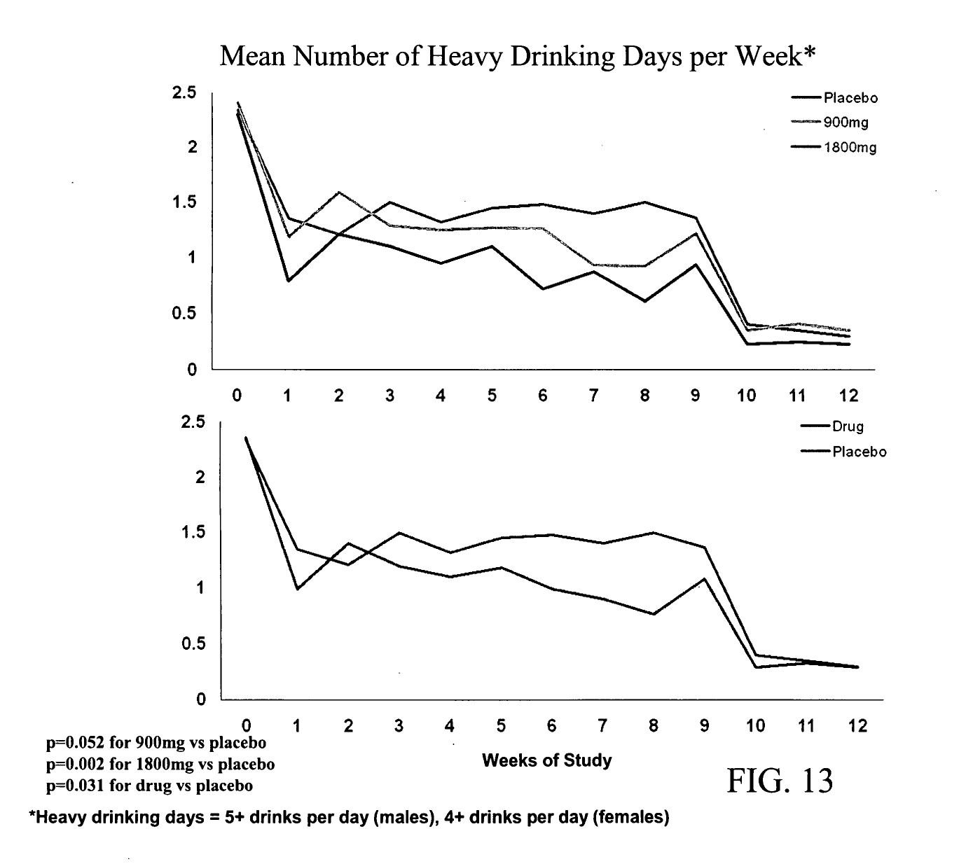

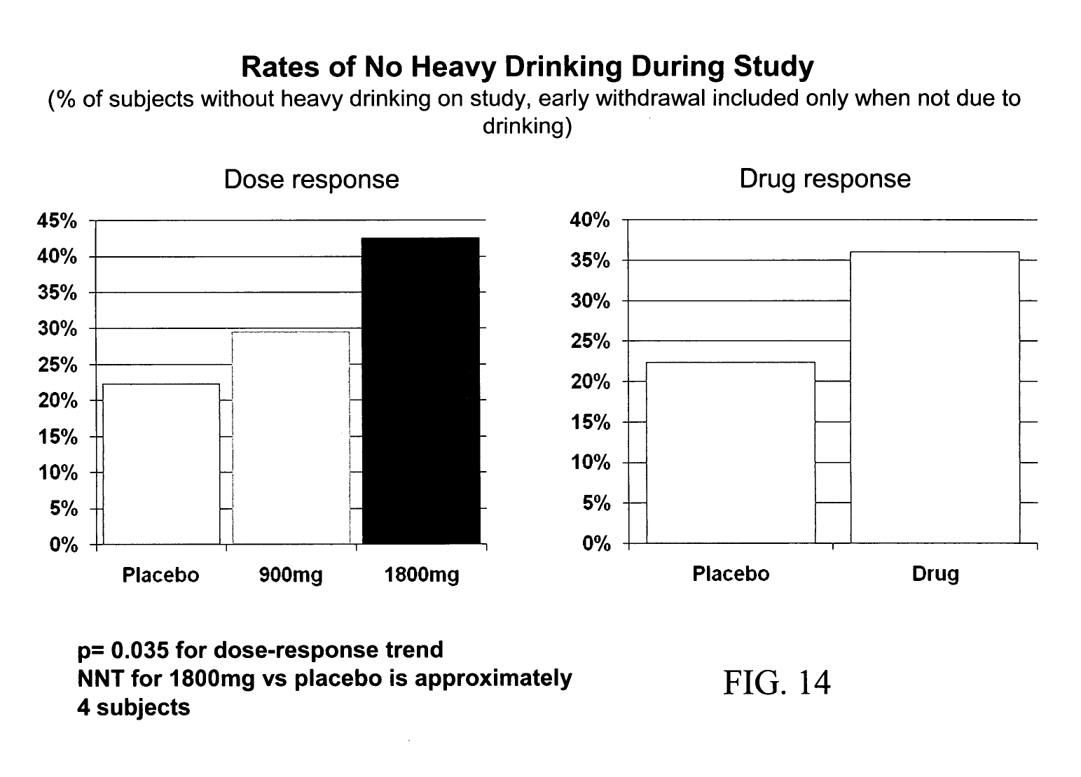

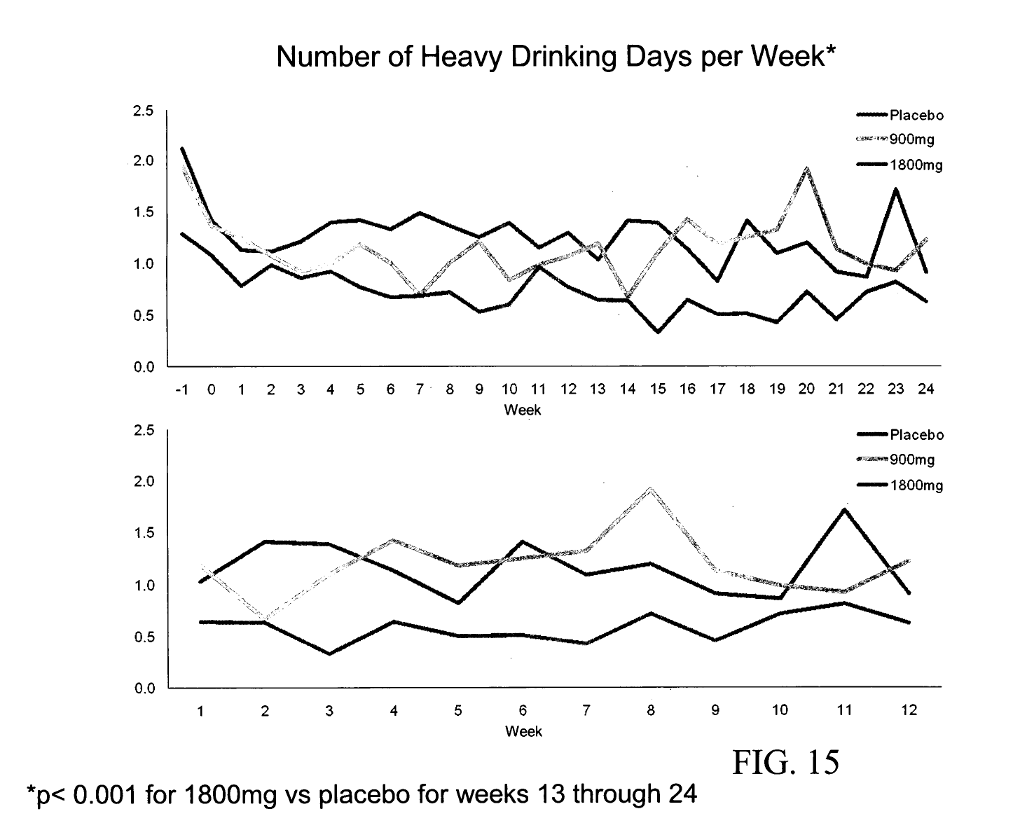

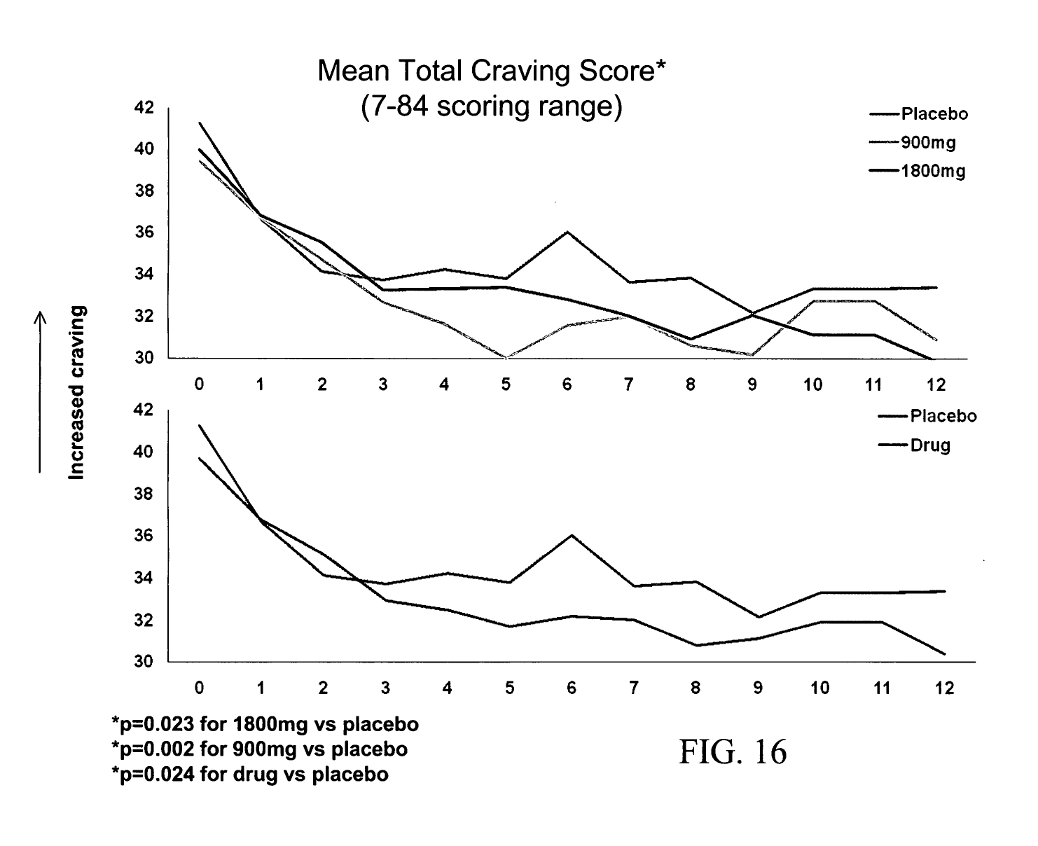

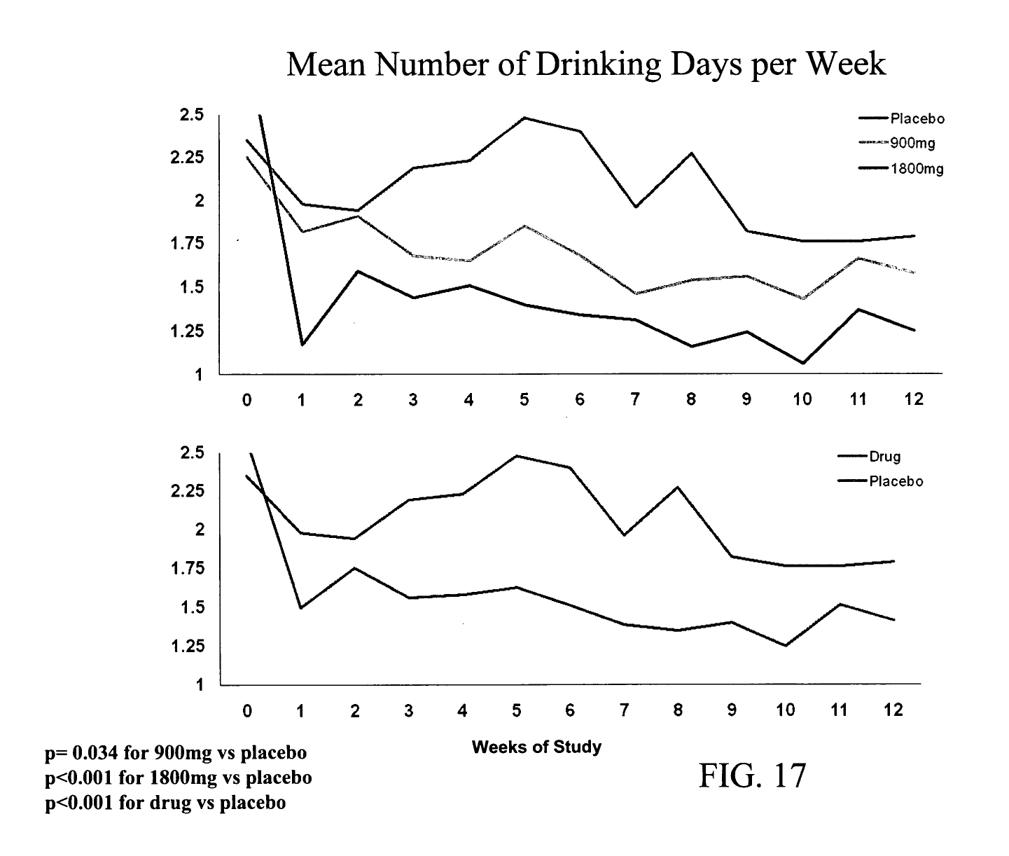

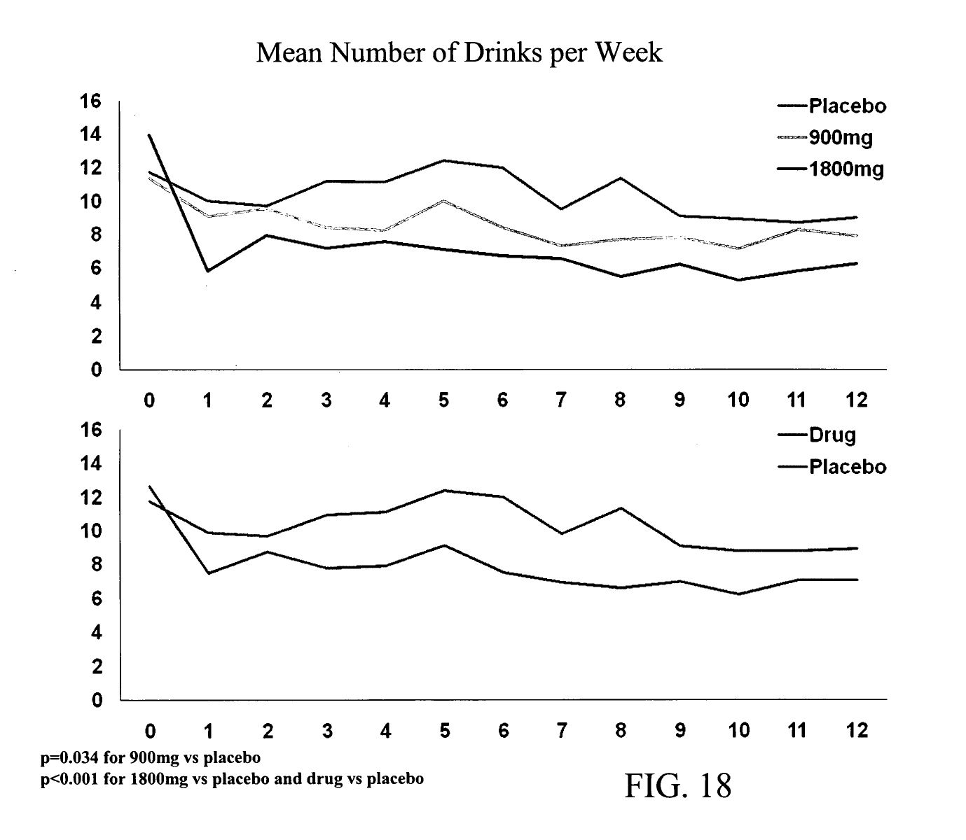

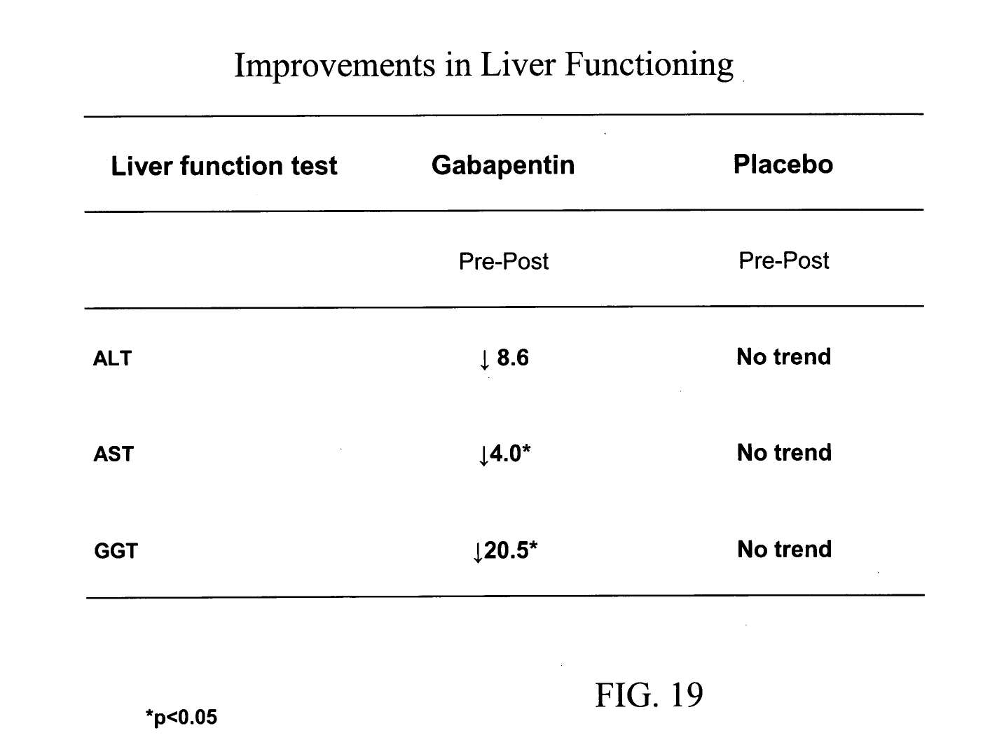

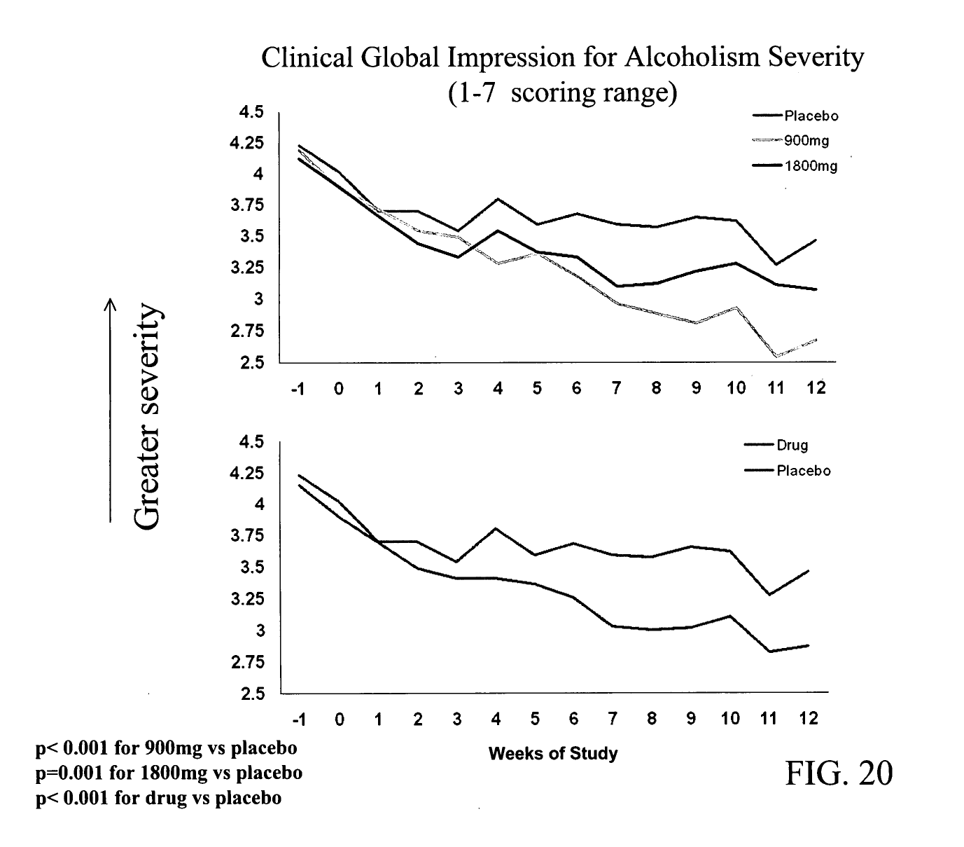

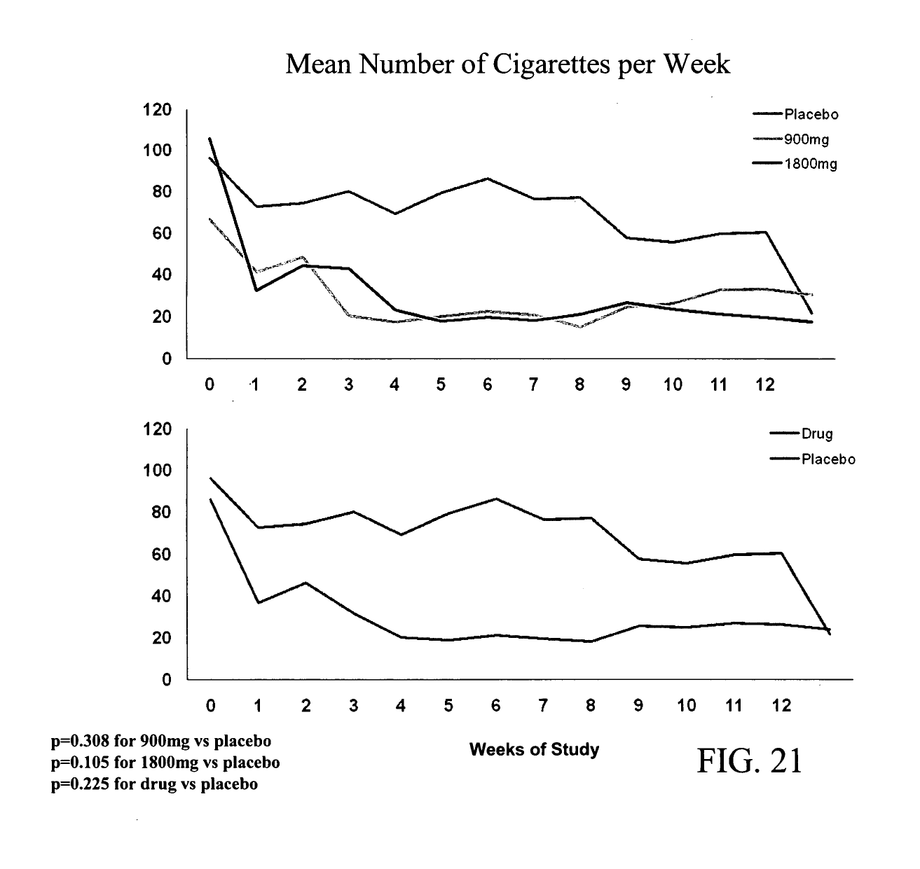

Subsequent to the interim analyses noted above, study of the entire subject pool (sample n=150) was completed. In addition to confirming the findings obtained from the interim analyses, results from the complete study additionally revealed that, compared to placebo group, subjects receiving gabapentin treatment displayed an increased number of abstinent days per week (FIG. 11), and increased rate of complete abstinence over the whole treatment period (FIG. 12). In addition, the treated subjects have a decreased number of heavy drinking days (FIG. 13) and an increased rate of no heavy drinking over the whole treatment period (FIG. 14). It was found that the beneficial effect was sustained for up to 12 weeks after completing treatment (FIG. 15). Further, data from the complete study showed a decreased craving (FIG. 16), decreased drinking frequency (FIG. 17) and quantity (FIG. 18), and improved liver functioning in the treated subjects (FIG. 19). The results indicate that gabapentin treatment leads to a decrease in overall severity of alcoholism (FIG. 20). These results are statistically significant in favor of gabapentin over placebo. Moreover, subjects receiving gabapentin also showed a dramatic reduction in the number of cigarettes smoked with gabapentin relative to (FIG. 21), although the data is not statistically significant due to the small number of smokers in the subject pool. Overall, results from the complete study demonstrated that gabapentin helped some people to not drink at all (more so than placebo), and that, among those who did drink, they did so at a less pathological level with gabapentin treatment relative to subjects taking placebo. The latter treatment effect is clearly reflected in the improvement in liver functioning and overall alcoholism severity.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, patents, and patent applications cited in this specification are herein incorporated by reference as if each was specifically and individually indicated to be incorporated by reference.

What is claimed is:

1. A method for treating alcohol dependence and preventing relapse, comprising administering to a subject following acute alcohol withdrawal a therapeutically effective amount of gabapentin, or an analog or pharmaceutically acceptable salt thereof, thereby treating alcohol dependence and preventing relapse in the subject.

2. The method of claim 1, wherein the subject has been in abstinence from alcohol for at least about 1-4 days, 5-10 days, 10-45 days, 45-120, 120-180 days or longer.

3. The method of claim 1, wherein the subject suffers from symptoms of protracted abstinence.

4. The method of claim 3, wherein the symptoms of protracted abstinence are craving for alcohol, disturbance in sleep and negative affect.

5. The method of claim 1, wherein the subject is treated for a period that is at least 1 week, 1 month, 3 months, 6 months, 1 year, 2 years or longer.

6. The method of claim 1, wherein the subject is administered with a daily gabapentin dosage of between about 100 to about 5,000 mg.

7. The method of claim 1, wherein the subject is administered with a daily gabapentin dosage of between about 300 mg to about 2000 mg.

8. The method of claim 1, wherein the subject is administered with a daily gabapentin dosage of between about 900 mg to about 1800 mg.

9. The method of claim 1, wherein the analog is pregabalin.

10. A method for reducing alcohol craving or consumption by a subject with alcohol dependence, comprising administering to a subject following acute alcohol withdrawal a therapeutically effective amount of gabapentin, or an analog or pharmaceutically acceptable salt thereof, thereby reducing alcohol craving or consumption by the subject.

11. The method of claim 10, wherein the subject has been in abstinence from alcohol for at least about 1-5 days, 5-10 days, 10-45 days, 45-120, 120-180 days or longer.

12. The method of claim 10, wherein the subject suffers from symptoms of protracted abstinence.

13. The method of claim 12, wherein the symptoms of protracted abstinence are disturbance in sleep and disturbance in affect.

14. A method for treating or alleviating symptoms of protracted alcohol abstinence, comprising administering to a subject following acute alcohol withdrawal a therapeutically effective amount of gabapentin, or an analog or pharmaceutically acceptable salt thereof, thereby treating or alleviating symptoms of protracted abstinence in the subject.

15. The method of claim 14, wherein the symptoms are craving for alcohol, disturbance in sleep and negative affect.

16. The method of claim 14, wherein the subject has been in abstinence from alcohol for at least about 1-5 days, 5-10 days, 10-45 days, 45-120, 120-180 days or longer.

17. A method for treating cannabis dependence, comprising administering to a subject with cannabis dependence a therapeutically effective amount of gabapentin, or an analog or pharmaceutically acceptable salt thereof, thereby treating cannabis dependence of the subject.

18. The method of claim 17, wherein the subject is currently using cannabis.

19. The method of claim 18, wherein the treatment is to help or enable the subject to cease or reduce cannabis use.

20. The method of claim 17, wherein the subject has ceased or discontinued cannabis use.

21. The method of claim 20, wherein the treatment is to treat or alleviate symptoms of acute cannabis withdrawal.

22. The method of claim 17, wherein the subject is in protracted abstinence from cannabis use.

23. The method of claim 17, wherein the subject is administered with a daily gabapentin dosage of between about 300 to about 2000 mg.

24. The method of claim 17, wherein the subject is administered with a daily gabapentin dosage of between about 900 mg to about 1800 mg.

25. The method of claim 17, wherein the analog is pregabalin.
